(19) United States
(12) Patent Application Publication
(10) Pub. No.: US 2011/0118183 A1
(43) Pub. Date: May 19, 2011

(54) N-GLYCOSYLATED HUMAN GROWTH HORMONE WITH PROLONGED CIRCULATORY HALF-LIFE

(75) Inventors: Gert Bolt, Vaerlose (DK); Claus Kristensen, Bronshoj (DK); Esper Boel, Lyngby (DK); Thomas Veje Lundgaard, Gentofte (DK)

(73) Assignee: Novo Nordisk Health Care AG, Zurich (SE)

(21) Appl. No.: 13/000,266

(22) PCT Filed: Jun. 26, 2009
(86) PCT No.: PCT/EP2009/058055

§ 371 Date: Feb. 1, 2011

(30) Foreign Application Priority Data
Jun. 27, 2008 (EP) 08159273.5

(51) Int. Cl. A61K 38/27; C07K 14/61; C12P 21/02; A61P 5/06

(52) U.S. Cl. 514/11.4; 530/399; 435/69.4

(57) ABSTRACT

The present invention relates to novel human growth hormone (hGH) variant(s) with one or more N-glycans. The hGH variants of the invention comprises an amino acid sequence which includes at least one N-glycosylation motif (N-X-S/T) arising from one or more mutations not present in the wild type hGH. The hGH variants of the invention have a prolonged circulatory half-life and thus can be effectively used as a protein therapeutic for disease states that will benefit from increased levels of hGH. The process of obtaining the hGH variants is also encompassed by the invention.

### FIELD OF THE INVENTION

The present invention relates to novel human growth hormone (hGH) variant(s) with at least one N-glycosylation motif (N-X-S/T), which N-glycosylation motif(s) are not present in the wild type hGH. The hGH variants of the invention have a prolonged circulatory half-life for use as protein therapeutic for disease states that will benefit from increased levels of hGH.

### BACKGROUND OF THE INVENTION

Human growth hormone (hGH) is a protein of 191 amino acids length with two disulphide bridges and a molecular weight of 22 kDa. The disulphide bonds link positions 53 and 165 and positions 182 and 189. hGH plays a key role in promoting growth, maintaining normal body composition, anabolism and lipid metabolism (Barnels K, Keller U. Clin. Endocrinol. Metab. 10, 337 (1996)). It also has direct effects on intermediate metabolism, such as decreased glucose uptake, increased lipolysis, increased amino acid uptake and protein synthesis. The hormone also exerts effects on other tissues including adipose tissue, liver, intestine, kidney, skeleton, connective tissue and muscle. Recombinant hGH has been produced and commercially available as, for ex: Genotropin™ (Pharmacia Upjohn), Nutropin™ and Protropin™ (Genentech), Humatrope™ (Eli Lilly), Serostim™ (Serono) and Norditropin™ (Novo Nordisk). Additionally, an analogue with an additional methionine residue at the N-terminal end is also marketed as, for ex: Somatonorm™ (Pharmacia Upjohn/Pfizer).

In general, subnormal levels of hGH leads to growth-related deficiencies. For example, hGH maintains normal body composition by increasing nitrogen retention and stimulation of skeletal muscle growth. Growth hormone deficiency in children leads to dwarfism which can be effectively treated by exogenous administration of hGH. It is also believed that the declining levels of hGH may be responsible for manifestations of ageing which includes decreased lean body mass, expansion of adipose tissue mass and shrinking of the skin.

The current hGH therapeutic regimen requires daily subcutaneous injections. A dosing regimen with fewer injections per week would be beneficial. Several principles for increasing the half-life of proteins have been discovered, but their applicability varies among different proteins, partly because different proteins are cleared by different routes and mechanisms.

Some proteins can achieve an increased half-life by the addition of N-glycans at amino acid positions that are not glycosylated in the wild-type protein (Sinclair and Ellliott, J Pharm Sci. 94, 1626 (2005)). N-glycans are attached to proteins by eukaryotic cells producing the protein. The cellular N-glycosylation machinery of eukaryotic cells recognizes N-X-S/T motifs and adds a glycan at the N residue of this motif, as the nascent protein is translocated from the ribosome to the endoplasmic reticulum (Kiely et al. J Biol. Chem. 251 5490 (1976); Glabe et al. J Biol. Chem. 255, 9236 (1980)). Thus glycoengineered proteins can be produced by introducing mutations that add N-glycosylation sites to the amino acid sequence of the protein. This principle has been employed to obtain longer-acting second generation erythropoietin (Aranesp®, Amgen), Elliott et al. Nature Biotechnology 21, 414 (2003).

### SUMMARY OF THE INVENTION

The present invention concerns human growth hormone (hGH) variant(s) with at least one N-glycosylation motif (N-X-S/T), which N-glycosylation motifs) are not present in the wild type hGH. In one embodiment, said variants are expressed in eukaryotic cells, which provides N-glycosylation at said sites, leading to the expression of hGH variants with a prolonged circulatory half-life. Such hGH variants are useful as protein therapeutics for disease states that will benefit from increased levels of hGH, particularly in treatments with less than daily injections, for instance weekly injections.

### BRIEF DESCRIPTION OF THE DRAWINGS

FIG. 1A is the nucleotide sequence and deduced amino acid sequence of DNA encoding wild-type human growth hormone for expression in mammalian cells as described in example 1.

FIG. 1B is the protein sequence of mature hGH (SEQ ID NO:1).

FIG. 2 shows the yields of recombinant wild type human growth hormone measured by ELISA in the medium of HEK293 cells transiently transfected with pGB039 (example 2).

FIG. 3 shows the results of a BAF3-GHR cell assay with medium from HEK293 cells transiently transfected with constructs encoding human growth hormone variants with a N-glycosylation site that is utilized. Recombinant human growth hormone produced in bacteria served as standard and was tested in parallel. The human growth hormone variants tested were diluted to 10 nM, 3 nM, 1 nM, 100 ρM, 300 ρM, 30 ρM, 10 ρM, 3 ρM, 1 ρM, 0.3 ρM, and 0.1 ρM. Trendlines describing the logarithmic hGH concentration vs. the growth response using a variable slope were calculated with the GraphPad software (Prism) (Example 5).

FIG. 4 shows the results of BAF3-GHR cell assay with medium from HEK293 cells transiently transfected with constructs encoding human growth hormone variants with more than one N-glycosylation site. Recombinant human growth hormone produced in bacteria served as standard and was tested in parallel. The human growth hormone variants were tested diluted to 10 nM, 5 nM, 1 nM, 500 ρM, 100 ρM, 50 ρM, 10 ρM, 5 ρM, 1 ρM, 0.5 ρM and 0.1 ρM. Trendlines describing the logarithmic hGH concentration vs. the growth response using a variable slope were calculated with the GraphPad software (Prism) (Example 7).

FIG. 5 shows the mean human growth hormone concentration versus time in plasma of male Sprague Dawley rats injected intravenously with N-glycosylated human growth hormone variant L93N+A98N+L101T+G104N (TVL20) or with wild-type human growth hormone (Example 10).

FIG. 6 shows the results of a BAF3-GHR cell assay with medium from HEK293 cells transiently transfected with constructs encoding human growth hormone variants with a N-glycosylation site that is utilized. Recombinant human growth hormone produced in bacteria served as standard and was tested in parallel. The human growth hormone variants tested were diluted to 10 nM, 5 nM, 1 nM, 500 ρM, 100 ρM, 50 ρM, 10 ρM, 5 ρM, 1 ρM, 0.5 ρM, and 0.1 ρM. Trendlines describing the logarithmic hGH concentration vs. the growth response using a variable slope were calculated with the GraphPad software (Prism) (Example 13).

FIG. 7 shows the results of a BAF3-GHR cell assay with medium from HEK293 cells transiently transfected with constructs encoding human growth hormone variants with a N-glycosylation site that is utilized. Recombinant human growth hormone produced in bacteria served as standard and was tested in parallel. The human growth hormone variants tested were diluted to 10 nM, 5 nM, 1 nM, 500 ρM, 100 ρM, 50 ρM, 10 ρM, 5 ρM, 1 ρM, 0.5 ρM, and 0.1 ρM. Trendlines describing the logarithmic hGH concentration vs. the growth response using a variable slope were calculated with the GraphPad software (Prism) (Example 13).

FIG. 8 shows the results of BAF3-GHR cell assay with medium from HEK293 cells transiently transfected with constructs encoding human growth hormone variants with more than one N-glycosylation site. Recombinant human growth hormone produced in bacteria served as standard and was tested in parallel. The human growth hormone variants were tested diluted to 10 nM, 5 nM, 1 nM, 500 ρM, 100 ρM, 50 ρM, 10 ρM, 5 ρM, 1 ρM, 0.5 ρM and 0.1 ρM. Trendlines describing the logarithmic hGH concentration vs. the growth response using a variable slope were calculated with the Graph Pad software (Prism) (Example 15).

FIG. 9 shows the mean human growth hormone concentration versus time in plasma of male Sprague Dawley rats injected intravenously with N-glycosylated human growth hormone variant Q49N+E65N+G104N+R127N+E129T (TVL64), Q49N+E65N+S71N+L73T+G104N+R127N+E129T (TVL66), or Q49N+E65N+S71N+L73T+L93N+G104N+R127N+E129T (TVL67) or with wild-type human growth hormone (Example 17).

FIG. 10 shows the mean human growth hormone concentration versus time in plasma of male Sprague Dawley rats injected subcutaneously with N-glycosylated human growth hormone variant Q49N+E65N+G104N+R127N+E129T (TVL64), Q49N+E65N+S71N+L73T+G104N+R127N+E129T (TVL66), or Q49N+E65N+S71N+L73T+L93N+G104N+R127N+E129T (TVL67) or with wild-type human growth hormone (Example 17).

### DESCRIPTION OF THE INVENTION

The present invention relates to human growth hormone (hGH) variant(s) with prolonged half-life, which can be used for therapeutic purposes, and provides recombinantly expressed human growth hormone variants carrying additional N-glycosylations, said variant comprising an amino acid sequence comprising one or more N-glycosylation motifs (N-X-S/T), which are not present in the wild type human growth hormone. The nucleic acid hGH is mutated at specific amino acid positions and the recombinant expression of the nucleic acid in a eukaryotic cell will yield N-glycosylated derivatives of these hGH variants having a prolonged circulatory half-life as compared to the wild type hGH. Due to its improved pharmacokinetic properties, the hGH variants of the invention are more useful as a therapeutic for disease states that will benefit from increased levels of hGH, as it decreases the dosing frequency as compared to unaltered hGH.

In the present context, the term “variant” is intended to refer to either a naturally occurring variation of a given polypeptide or a recombinantly prepared or otherwise modified variation of a given peptide or protein, such as human growth hormone (the sequence of which is presented in SEQ ID No. 1), in which one or more amino acid residues have been modified by amino acid substitution, addition, deletion, insertion or invertion. For clarification, a hGH variant may also be derivatized or otherwise modified, i.e., by covalent attachment of any type of molecule to the parent polypeptide. Typical modifications may be attachment of amides, carbohydrates, alkyl groups, acyl groups, esters, PEGylations and the like to a polypeptide comprising the human growth hormone variant sequence. In particular, a hGH variant may also carry N-glycosylation. The hGH varian may additionally comprises further mutations, not linked to introduction of N-glycosylation sites not present in wild type human growth hormone. Such additional mutations may be included for a variety of reasons, such as to enable modification by covalent attachment of any type of molecule as described above.

In one embodiment, the invention provides a hGH variant which is a polypeptide comprising an amino acid sequence having at least 80%, such as at least 85%, for instance at least 90%, such as at least 95%, for instance 100% identity with the amino acid sequence in SEQ ID No. 1.

The term “identity” as known in the art, refers to a relationship between the sequences of two or more peptides, as determined by comparing the sequences. In the art, “identity” also means the degree of sequence relatedness between peptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two peptides for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp. 3 (1978) for the PAM 250 comparison matrix; Henikoff et al., Proc. Natl. Acad. Sci USA 89, 10915-10919 (1992) for the BLOSUM 62 comparison matrix) is also used by the algorithm.

Preferred parameters for a peptide sequence comparison include the following:

Algorithm: Needleman et al., J. Mol. Biol. 48, 443-453 (1970); Comparison matrix: BLOSUM 62 from Henikoff et al., PNAS USA 89, 10915-10919 (1992); Gap Penalty: 12, Gap Length Penalty: 4, Threshold of Similarity: 0.

The GAP program is useful with the above parameters. The aforementioned parameters are the default parameters for peptide comparisons (along with no penalty for end gaps) using the GAP algorithm.

In one embodiment, the hGH variant comprises amino acid sequence with at least one N-glycosylation motif (N-X-S/T) arising from one or more mutations selected from the group consisting of S55N, Q69N, E74S, E74T, R77N, I83N, L93N, A98N, L101S, L101T, G104N, S106N, Y111S, Y111T, I121N, D130N, K140N, T142N, G161S, G161T and E186N.

In one embodiment, the hGH variant comprises amino acid sequence with at least one N-glycosylation motif (N-X-S/T) arising from one or more mutations selected from the group consisting of Q69N, R77N, I83N, L93N, A98N, L101T, G104N, S106N, Y111T, I121N, D130N, K140N, G161T, and E186N.

The invention also encompasses hGH variant comprising amino acid sequence with at least one N-glycosylation motif (N-X-S/T) arising from one or more of the following sets of mutations:

L93N, A98N, L101T and G104N;

L93N, A98N, and G104N; or

L93N, L101T, and G104N.

In one embodiment, the invention provides an isolated nucleic acid sequence encoding a hGH variant, wherein the said variant comprises an amino acid sequence which includes at least one N-glycosylation motif (N-X-S/T) arising from one or more mutations not present in the wild type hGH.

The invention provides an isolated nucleic acid sequence encoding a hGH comprising at least one N-glycosylation motif (N-X-S/T) arising from one or more mutations selected from the group consisting of S55N, Q69N, E74S, E74T, R77N, I83N, L93N, A98N, L101S, L101T, G104N, S106N, Y111S, Y111T, I121N, D130N, K140N, T142N, G161S, G161T and E186N.

The invention also provides an isolated nucleic acid sequence encoding a hGH comprising at least one N-glycosylation motif (N-X-S/T) arising from one or more mutations selected from the group consisting of Q69N, R77N, I83N, L93N, A98N, L101T, G104N, S106N, Y111T, I121N, D130N, K140N, G161T, and E186N.

Furthermore, the invention provides isolated nucleic acid sequence encoding a hGH variant comprising amino acid sequence with N-glycosylation motifs (N-X-S/T) arising from one or more of the following sets of mutations:

L93N, A98N, L101T and G104N;

L93N, A98N, and G104N; or

L93N, L101T, and G104N.

Additionally, the invention provides an eukaryotic host cell comprising the vector consisting a nucleic acid encoding a human growth hormone variant comprising an amino acid sequence which includes at least one N-glycosylation motif (N-X-S/T) arising from one or more mutations not present in the wild type human growth hormone.

The invention also encompasses a vector comprising a nucleic acid encoding a human growth hormone variant with N-glycosylation motifs (N-X-S/T) arising from one or more of the following sets of mutations:

L93N, A98N, L101T and G104N;

L93N, A98N, and G104N; or

L93N, L101T, and G104N.

In one embodiment the invention provides an N-glycosylated human growth hormone variant, which is glycosylated in one or more N-glycosylation motif(s) arising from one or more mutations as described herein above.

Furthermore, the invention provides a pharmaceutical composition comprising a human growth hormone variant comprising an amino acid sequence which includes at least one N-glycosylation motif (N-X-S/T) arising from one or more mutations not present in the wild type human growth hormone and a pharmaceutically acceptable carrier. The aforesaid pharmaceutical composition encompasses any of the different hGH variants described in the current disclosure.

In one embodiment, the invention provides a method of treating a mammal in need of human growth hormone, said method comprising administering to the mammal therapeutically effective amount of any of the human growth hormone variants described in the current disclosure.

The process of obtaining an N-glycosylated hGH variant comprising at least one N-glycosylation motif (N-X-S/T) arising from one or more mutations not present in wild type hGH, said process comprising the steps of: (a) transfecting a cell capable of performing N-glycosylation and capable of expressing said mutant human growth hormone with a nucleic acid encoding the said variant human growth hormone; and (b) expressing said variant human growth hormone is also encompassed in the present invention.

The present invention provides human growth hormone (hGH) variants comprising an amino acid sequence which includes at least one N-glycosylation site at specific amino acid positions; the hGH variants of the invention are therapeutically active and have pharmacokinetic parameters and properties that are improved relative to wild type hGH protein that is not glycosylated.

In one embodiment, the hGH variant of the present invention is the product of the expression of an exogenous DNA sequences that has been transfected into an eukaryotic host cell. For example, the hGH of the present invention is recombinantly produced. Production of recombinant hGH is well known in the art and can be readily recognized by a person skilled in the art (Ex: U.S. Pat. No. 4,670,393).

In one embodiment, the present invention provides a recombinant hGH with appropriate site or sites in the polypeptide to achieve an active N-glycosylated protein with improved circulatory half-life as compared to the wild type hGH. The invention as described herein is conveniently performed by using recombinant DNA technology.

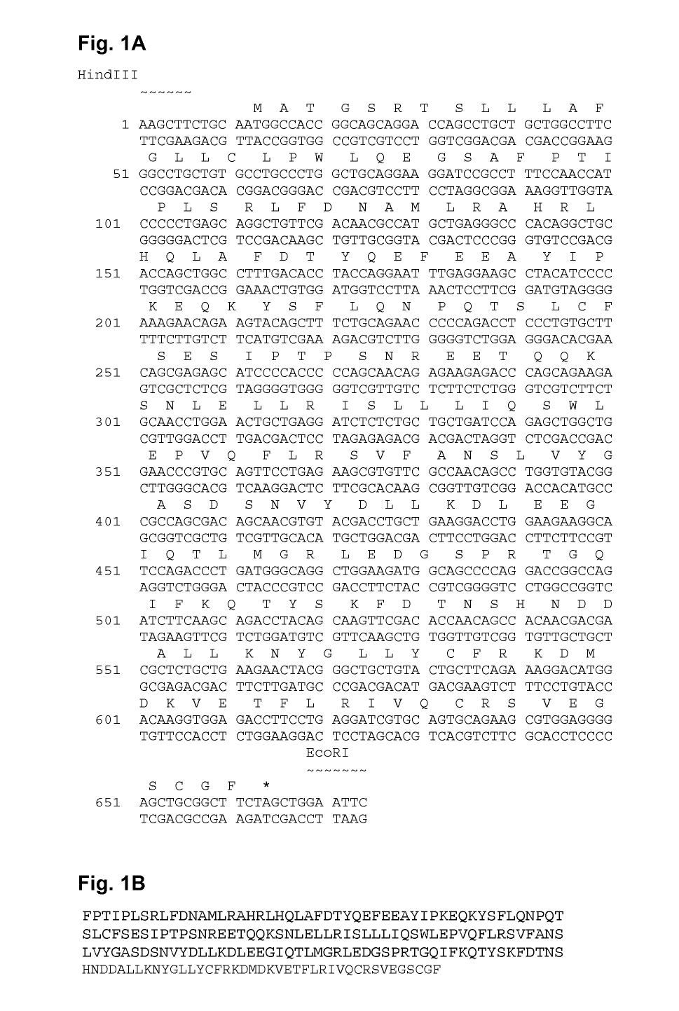

In general, the DNA sequence encoding hGH is cloned and manipulated so that it can be expressed in a convenient host. The nucleotide sequence shown in FIG. 1A encodes the 217 amino acid hGH preprotein (SEQ ID NO 65 and 66). The N-terminal 26 amino acids constitute the signal peptide, which is cleaved off intracellularly, when hGH is produced in eukaryotic cells. Thus, mammalian cells expressing the human growth hormone encoded by the sequence shown in FIG. 1A secrete the mature 191 amino acid growth hormone (SEQ ID NO 1) provided in FIG. 1B.

The hGH DNA is inserted into an appropriate plasmid or vector that is used to transform or transfect a host cell. Prokaryotes, eukaryotic organisms, such as yeast cultures, or cells derived from multicellular organisms, are used in the art for cloning and expressing DNA sequences.

Appropriate host cells for use according to the present invention are cells, which are capable of N-glycosylation. N-glycosylation is the addition of a saccharide moiety to the asparagine residue in N-X-S/T motifs. Such a saccharide moiety attached by the N-glycosylation machinery of eukaryotic cells to the amide nitrogen in the side-chain of asparagine is called an N-glycan.

Eukaryotic cells, such as mammalian cells, are generally capable of performing such N-glycosylation. Examples of cell lines, which are suitable for use in the present invention are the Chinese hamster ovary (CHO) (ATCC CCL 61), baby hamster kidney (BHK) and 293 (ATCC CRL 1573; Graham et al., J. Gen. Virol. 36:59-72, 1977) cell lines. In addition, a number of other cell lines may be used within the present invention, including Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1), COS-1 (ATCC CRL 1650), DUKX cells (Urlaub and Chasin, Proc. Natl. Acad. Sci. USA 77:4216-4220, 1980) and CHO-DG44 cells (Urlaub et al. Cell 33:405-412, 1983). In one embodiment, the host cell used for expressing hGH variants with N-glycosylation sites of the present invention is a mammalian cell. In one embodiment, the host cell used for expressing hGH variants with N-glycosylation sites of the present invention is a CHO cell.

Apart from mammalian cells engineered yeast cells can also be used to express glycosylated proteins by use of an appropriate system such as GlycoFi ( ).

Host cells used for the expression of hGH are cultured under conditions suitable for cell growth and for expression of the hGH variant. In particular, the culture medium contains appropriate nutrients and growth factors suitable for the growth of the chosen host cell for the said purpose. Suitable culture conditions for mammalian host cells, for instance, are described in Mammalian Cell Culture (Mather, J. P. ed., Plenum Press 1984) and Barnes and Sato, Cell, 22:649 (1980). More recently, animal component-free processes are increasingly becoming the standard for manufacturing biopharmaceuticals (Butler et al. Appl Microbiol Biotechnol 68:283, 2005). Furthermore, the chosen culture conditions should allow transcription, translation, and protein transport between cellular compartments. Some of the factors that affect these processes include but not limited to, for example, DNA/RNA copy number; factors that stabilize RNA; nutrients, supplements, and transcriptional inducers or repressors present in the culture medium; temperature, pH, and osmolality of the culture; and cell density. The manipulation of the aforesaid factors to promote adequate expression in a particular vector-host cell system is readily recognizable for a person skilled in the art.

Plasmid vectors containing replication and control sequences that are derived from compatible species with the host cell are generally used for expression. The vector carries a replication site, as well as sequences that encode protein of interest that are capable of providing phenotypic selection in transformed cells.

Following cloning of the hGH gene, different techniques can be used to produce the variant DNA that encodes for modified amino acid sequence. These techniques include site-specific mutagenesis (Carter et al., Nucl Acids Res. 13:4331, 1986; Zoller et al. Nucl Acids Res. 10:6487, 1987), cassette mutagenesis (Wells et al. Gene, 34:315, 1985), restriction selection mutagenesis (Wells et al. Philos Trans R Soc. London SerA, 317: 415, 1986), or other known techniques that are recognized by a person skilled in the art. In a preferred embodiment, site-specific mutagenesis was used in the present invention to produce the hGH with the glycosylation sites. When operably linked to an appropriate expression vector, glycosylation site hGH variants are obtained. Human growth factor (hGH) variants can also be obtained by expressing and secreting such molecules from the expression host by use of an appropriate signal sequence operably linked to the DNA sequence encoding the hGH parent or variant. Such methods are well known to those skilled in the art. The present invention also includes other methods that can be employed to produce hGH polypeptides such as the in vitro chemical synthesis of the desired hGH variant (Barany et al. in The Peptides, eds. E. Gross and J. Meienhofer, Academic Press: New York 1979, Vol. 2, pp. 3-254).

Carbohydrates are attached to glycopeptides in several ways of which N-linked to asparagine and O-linked to serine and threonine are the most relevant for recombinant glycoprotein therapeutics. A determining factor for initiation of glycosylation of a protein is the primary sequence context, although clearly other factors including protein region and conformation have their roles. N-linked glycosylation occurs at the consensus sequence N-X-S/T, where X can be any amino acid other than proline. In a preferred embodiment, N-glycosylation sites formed by amino acid substitutions involving cysteine or proline residues were disregarded.

The hGH analogs described herein comprise an amino acid sequence which includes at least one additional site for glycosylation as compared to the wild type hGH which is not glycosylated. The site for introducing N-glycosylation in a polypeptide can be located anywhere in the sequence. To prevent interference with the protein structure or folding the one or more N-glycosylation site(s) is in an embodiment selected to be on the surface of the protein. Furthermore, interfering with binding to the growth hormone receptor is also undesirable thus introduction of N-glycosylation sites on the binding interphase of human growth hormone is undesirable. In an embodiment the one or more N-glycosylation motifs are introduced in one or several regions of the mature human growth hormone protein. In one embodiment at least one N-glycosylation motif (N-X-S/T) arises from one or more mutations in amino acid residues 49-75, 93-104 and 111-140 of the mature hGH (SEQ ID NO 1). In further embodiments of the invention at least two, at least three, at least four or all N-glycosylation motifs are introduced in amino acid residues 49-75, 93-104 and 111-140. In one embodiment all N-glycosylation motifs are introduced in amino acid residues 49-77, 93-104 and 127-133.

In one embodiment, the present invention involves human growth hormone (hGH) comprising amino acid sequence with at least one N-glycosylation motif (N-X-S/T) arising from one or more mutations of the wild type hGH. An N may be introduce in the appropriate distance from an S or T present in wild type or an S or T (denoted S/T in the following may be introduce in the appropriate distance to and N present in wild type. Alternatively a N-glycosylation motif can be generate by introduction both an N and a S or T.

In one embodiment, the invention provides a hGH variant comprising an amino acid sequence with one or more N-glycosylation motifs (N-X-S/T) arising from one or more single mutations or double mutations selected from the group of mutation(s)/mutation pair(s) consisting of: K41N, Q49N, S55N, E65T, E65S, E65N, Q69N, E74S, E74T, R77N, I83N, L93N, A98N, L101S, L101T, G104N, S106N, Y111S, Y111T, I121N, D130N, P133N, K140N, T142N, G161S, G161T, E186N, R19N+H21S/T, A34N+1365/T, L45N+N47S/T, 158N+P59F, S62N+R64S/T, S71 N+L73S/T, K115N+L117S/T, R127N+E129S/T, L128N+D130S/T and T175N+L177S/T.

In one embodiment, the invention provides a hGH variant comprising an amino acid sequence with one or more N-glycosylation motifs (N-X-S/T) arising from one or more single mutations or double mutations selected from the group of mutation(s)/mutation pair(s) consisting of: K41N, Q49N, E65T, E65N, Q69N, E74T, R77N, I83N, L93N, A98N, L101T, G104N, S106N, Y111T, I121N, D130N, P133N, K140N, T142N, T148N, G161T, E186N, R19N+H215, A34N+136S, L45N+N47S, 158N+P59F, S62N+R64T, S71 N+L73T, K115N+L117T, R127N+E129T, L128N+D130T and T175N+L177S.

In one embodiment, the invention provides a hGH variant comprising an amino acid sequence with one or more N-glycosylation motifs (N-X-S/T) arising from one or more single mutation(s) or double mutation(s) selected from the group of mutation(s)/mutation pair(s) consisting of: K41N, Q49N, E65T, E65N, E74T, L93N, A98N, L101T, G104N, Y111T, P133N, K140N, T142N, G161T, E186N, R19N+H21S, 158N+P59F, S62N+R64T, S71N+L73T, R127N+E129T and L128N+D130T. As seen in table 3, 9 and 10 these mutations gave rise to functional N-glycosylation motifs when expressed in HEK293 cells demonstrated by detection of a band with reduced mobility compared to wild type un-glycosylated hGH.

In one embodiment, the invention provides hGH variant comprising an amino acid sequence with one or more N-glycosylation motifs (N-X-S/T) arising from one or more of the following mutations:

### S55N, Q69N, E74S, E74T, R77N, I83N, L93N, A98N, L101S, L101T, G104N, S106N, Y111S, Y111T, I121N, D130N, K140N, T142N, G161S, G161T, and E186N.

In one embodiment, the hGH variant comprises an amino acid sequence with at least one N-glycosylation motif (N-X-S/T) arising from one or more of the following mutations: Q69N, R77N, I83N, L93N, A98N, L101T, G104N, S106N, Y111T, I121N, D130N, K140N, G161T, and E186N.

In one embodiment the invention provides a human growth hormone variant, comprising at least one N-glycosylation motifs (N-X-S/T) not present in the wild type human growth hormone that have been generated by introducing one or more mutation(s) selected from the group of mutation(s)/mutation pair(s) consisting of: Q49N, E65N, L93N, A98N, L101T, G104N , S71N+L73T and R127N+E129T.

In one embodiment, the hGH variant comprises an amino acid sequence with at least one N-glycosylation motifs (N-X-S/T) arising from one or more of the following mutations:

L93N, A98N, L101T and G104N;

L93N, A98N and G104N; or

L93N, L101T and G104N.

In one embodiment the human growth hormone variant comprise at least one of said N-glycosylation motifs (N-X-S/T) been generated by introducing: a) one or more mutations selected from the group of: Q49N, E65N, L93N, A98N, G104N and/or b) one or more double mutations selected from the group consisting of: S71N+L73T and R127N+E129T. Such individual embodiments comprise human growth hormone variants including the following set of mutations:

a) Q49N and R127N+E129T,

b) Q49N, E65N and G104N,

c) Q49N, L93N and R127N+E129T,

d) Q49N, E65N, L93N and G104N,

e) Q49N, E65N, G104N and R127N+E129T,

f) Q49N, E65N, S71N+L73T, G104N and R127N+E129T,

g) Q49N, E65N, S71N+L73T, L93N, G104N and R127N+E129T,

h) Q49N, E65N, S71N+L73T, L93N, A98N, G104N and R127N+E129T,

i) S71N+L73T, L93N, A98N and G104N,

j) L93N, G104N and R127N+E129T and

k) S71N+L73T, L93N, G104N and R127N+E129T.

In a further embodiment the hGH variant comprises modification(s) and/or secondary mutation(s) in addition to the mutation(s) generating N-glycosylation motif(s) (N-X-S/T) not present in the wild type human growth hormone as described herein above.

In one embodiment of the present invention the growth hormone compound is chemically modified via attaching moieties such as, but not limited to, PEGs, carbohydrates, albumin binders, fatty acids, alkyl chains, lipophilic groups, vitamins, bile acids, or spacers to the side chains or main chain of the growth hormone compound. Such modifications may be attached to an amino acid residue of the wild type human growth hormone sequence of to an amino acid residue inserted by substitution of an amino acid of the wt sequence.

Additional mutations of the hGH sequence giving rise to amino acid substitutions may also directly alter the functionality of the hGH variant. In one embodiment the hGH variant additionally comprise mutations that are resistant to proteolytic degradation such as described in EP534568 and WO2006048777. Mutations or modifications that are effectuated during expression in the host will abolish the need for subsequent modification steps in vitro and thereby shorten the productions process.

The hGH variant can be purified from culture medium by any method capable of separating the variant from components of the host cell or culture medium. Briefly the hGH variant is separated from the culture medium containing the host cells which will interfere with the further use of the variant, for example, in pegylation of the therapeutic hGH, or in its diagnostic use.

The general procedure for such separation allows the centrifugation or filtration of the culture medium or cell lysate to remove cellular debris. The supernatant is then typically concentrated or diluted to a desired volume or diafiltered into a suitable buffer to condition the preparation for further purification. Further purification of the hGH variant typically includes separating deamidated and clipped forms of the protein from the intact form.

Affinity chromatography; anion- or cation-exchange chromatography (using, e.g., DEAE SEPHAROSE); chromatography on silica; reverse phase HPLC; gel filtration (using, e.g., SEPHADEX G-75); hydrophobic interaction chromatography; metal-chelate chromatography; ultrafiltration/diafiltration; ethanol precipitation; ammonium sulfate precipitation; chromatofocusing; and displacement chromatography are some of the techniques known in the art that can be used for the purification of the hGH variant.

In one embodiment the invention provides an N-glycosylated human growth hormone variant, which is glycosylated in one or more N-glycosylation motif(s) generated by one or more mutations as described herein above.

Efficacy of the growth hormone is dependent on its interaction with growth hormone receptor (GHR). Thus the hGH variants that are produced are contacted with the GHR and the interaction, if any, between the receptor and each variant is determined for further analysis. These activities are compared to the activity of the wild-type hGH with the same receptor to determine which of the amino acid residues in the active domain are involved in the interaction with the receptor.

The interaction between the receptor and parent and variant is measured by any convenient in vitro or in vivo assay well known in the art. The in vitro assays can be used to determine any detectable interaction between a GHR and hGH. Such detection can include the measurement of colorimetric changes, changes in radioactivity, changes in solubility, proliferation inducing capacity, changes in molecular weight as measured by gel electrophoresis, and/or gel exclusion methods, etc. In vivo assays to detect physiological effects of hGH are for, ex, weight gain or change in electrolyte balance. In general, any in vitro or in vivo assay can be used so long as a variable parameter exists so as to detect a change in the interaction between the receptor and the hGH of interest. In a preferred embodiment, hGH variants produced by N-glycosylation in the present invention was examined for their proliferation inducing capacity on BAF3-GHR cells such as described in Example 5 and 13. The BAF3-GHR cells have previously been described in WO2006134148 which is incorporated herein by reference. BAF3-GHR cells are derived from the IL-3 dependent murine pro-B lymphoid BAF3 cell line. IL-3 activates JAK-2 and STAT, which are also activated by binding of growth hormone receptor binding. BAF3-GHR cells express the human growth hormone receptor, and responds with a dose-dependent proliferative response to stimulation with growth hormone.

Provided in the present invention is also a process for expressing a human growth hormone variant comprising a N-glycosylation site having steps including: (a) transfecting a cell capable of performing N-glycosylation and expressing said mutant human growth hormone with a nucleic acid encoding the said variant hGH; and (b) expressing said variant hGH.

In one embodiment, the cell is a eukaryotic cell, for ex: CHO cell. The vectors of the present invention may of course also be replicated in prokaryotic cells.

hGH variants comprising one or more N-glycosylation in N-glycosylation motifs not present in wild type human growth hormone may be differentiated from wt human growth hormone by use of several methods. The N-glycosylation(s) is likely to increase molecular weight of the variant compared to wild-type human growth hormone. Additionally or alternatively the N-glycosylation may affect the isoelectric point of a protein.

The “Isoelectric point” as used herein describes the pH at which the protein carries no net electric charge. Likewise, the isoelectric point of the individual amino acids in a protein is the pH at which the amino acid carries no net electric charge. An acidic amino acid has a neutral net electric charge at a pH below its isoelectric point, and a negative net electric charge at a pH above its isoelectric points. A basic amino acid has neutral net electric charge at a pH above its isoelectric point and a positive net electric charge at a pH below its isoelectric points. Thus, at any given pH the combined electric charges of the individual amino acids of a protein together with the charges of other moieties i.e. glycans determine the net electric charge of a protein. At a pH below their isoelectric point, proteins carry a net positive charge. At a pH above their isoelectric point, proteins carry a net negative charge. Sialic acids in glycan chains has acidic isoelectric points. Thus, addition of sialylated glycan chains to a protein induce a shift towards a more acidic isoelectric point. The isoelectric point of mature wild-type hGH is 5.27.

The isoelectric point of a protein is typically determined by isoelectric focusing. Isoelectric focusing is carried out by electrophoresis of the protein of interest in a medium with a pH gradient. When the protein reaches the region of the medium with the pH of the proteins isoelectric point, migration of the protein ceases, since the protein no longer has a net electric charge. Thus, the protein becomes focused into a sharp band at the pH of its isoelectric point. An example of the methodology is described by Eap and Baumann (Eap C B, Baumann P, Electrophoresis 9, 650 (1988)).

In one embodiment, the isoelectric point of a human growth hormone variant prepared by use of a method as described above is more acidic than the wild-type human growth hormone. In one embodiment the isoelectric point of a human growth hormone variant is less than 5.27, such as less than 5.0, such as less than 4.5 or such as less than 4.0. In one embodiment the isoelectric point of said human growth hormone variant is less than the isoelectric point of mature wild-type hGH, by such as more than 0.2 pH unit, or such as more than 0.4 pH unit, such as more than 0.6 pH unit, or such as more than 0.8 pH unit, such as more than 1.0 pH unit.

In one embodiment, a human growth hormone variant prepared by use of a method as described above has increased molecular weight compared to wild-type human growth hormone. An increase in molecular weight may be determined by one of several methods well known in the art, such as SDS-Page or mass spectrometry.

In one embodiment such weigh increase is due to the utilization of the N-glycosylation site(s) e.g. the addition of N-glycans to the human growth hormone variant. Mutations of the AA sequence may give rise to minor changes in molecular weight compared to wild type human growth hormone.

As N-glycans may be removed or modified enzymatically using a glycosidase such as PNGase F-enzyme or Neuraminidase-enzyme, the attribution of N-glycan(s) to the weight increase, may be confirmed by in vitro analysis.

In one embodiment a human growth hormone variant prepared by use of a method as described above changes mobility in an SDS-PAGE, when treated with a glycosidase. Detection of mobility shifts is generally known in the art. SDS-PAGE is frequently used and mobility shifts are easily detected as described in example 5 and 13. A mobility shift representing removal of one N-glycan will generally be in the order of 2-5 kDa, if more than one N-glycan is removed the mobility shift will increase accordingly. In one embodiment said glycosidase is PNGase F-enzyme or neuraminidase-enzyme. In one embodiment the shift is at least 1 kDa, such as at least 2 kDa, such as at least 3 kDa, such as at least 5 kDa or such as at least 10 kDa. In one embodiment the shift is 1-10 kDa or 2-6 kDa.

In an embodiment the invention relates to a preparation comprising an N-glycosylated human growth hormone variant, which N-glycosylated human growth hormone variant is a human growth hormone variant as described herein, which human growth hormone variant has been glycosylated with one or more N-glycans, wherein said N-glycan(s) has been attached to one or more of the N-glycosylation motif(s) (N-X-S/T) in said human growth hormone variant, which N-glycosylation motif(s) are not present in the wild type human growth hormone. In an embodiment of the invention such a preparation comprises at least 20% of the human growth hormone variant is N-glycosylated as estimated on an SDS-page gel. In further embodiments at least 25, such as 40, 50, 60, 80 or 90% of the human growth hormone variant in said preparation is N-glycosylated. In an embodiment a preparation comprising human growth hormone variant as described herein, 60-100% of said human growth hormone variant is N-glycosylated, such as 70-100%, such as 80-100%, such as 90-100% or such as 95-100%. Estimations of content of N-glycosylation are exemplified herein in examples 5 and 13, which may also be performed using appropriate scanning equipment known in the art. For growth hormone variants comprising more than one glycosylation motifs such a preparation may comprise human growth hormone variants with different numbers of N-glycans. In one embodiment all glycosylation motifs, not present in the wild type human growth hormone, are used. In one embodiment at least 20% of the human growth hormone variant comprised by a preparation included N-glycans attached to all of said glycosylation motifs, not present in the wild type human growth hormone. In one embodiment at least 25, such as at least 40, 50, 60, 80 or 90% of the human growth hormone variant in said preparation is N-glycosylated on all of said glycosylation motifs, not present in the wild type human growth hormone. In an embodiment according to the invention a preparation comprising N-glycosylation human growth hormone variant, 60-100%, such as 70-100%, such as 80-100%, such as 90-100% or such as 95-100% of said such human growth hormone variant is N-glycosylated on all of said glycosylation motifs, not present in the wild type human growth hormone.

Compounds of the present invention also exert growth hormone activity and may as such be used in the treatment of diseases or states which will benefit from an increase in the amount of circulating growth hormone. Such diseases or states include growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1^{st} toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Chron's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; or short stature due to glucocorticoid treatment in children. Growth hormones have also been used for acceleration of the healing of muscle tissue, nervous tissue or wounds; the acceleration or improvement of blood flow to damaged tissue; or the decrease of infection rate in damaged tissue, the method comprising administration to a patient in need thereof an effective amount of a therapeutically effective amount of a compound of formula I. The present invention thus provides a method for treating these diseases or states, the method comprising administering to a patient in need thereof a therapeutically effective amount of a growth hormone or growth hormone compound conjugate according to the present invention.

Typically, the amount of variant growth hormone administered is in the range from 10⁻⁷-10⁻³ g/kg body weight, such as 10⁻⁶-10⁻⁴ g/kg body weight, such as 10⁻⁵-10⁻⁴ g/kg body weight.

In one embodiment, the invention provides the use of a growth hormone or growth hormone compound conjugate in the manufacture of a medicament used in the treatment of the above mentioned diseases or states.

The hGH variants described herein is intended to be used as a therapeutic protein. The present invention is also directed to pharmaceutical compositions comprising a protein modified by any of the methods disclosed herein. In one aspect, such a pharmaceutical composition comprises a modified protein such as human growth hormone (hGH), which is present in a concentration from 10⁻¹⁵ mg/ml to 200 mg/ml, such as e.g. 10⁻¹⁰ mg/ml to 5 mg/ml and wherein said composition has a pH from 2.0 to 10.0. The composition may further comprise a buffer system, preservative(s), tonicity agent(s), chelating agent(s), stabilizers and surfactants. In one embodiment of the invention the pharmaceutical composition is an aqueous composition, i.e. composition comprising water. Such composition is typically a solution or a suspension. In a further embodiment of the invention the pharmaceutical composition is an aqueous solution. The term “aqueous composition” is defined as a composition comprising at least 50% w/w water. Likewise, the term “aqueous solution” is defined as a solution comprising at least 50% w/w water, and the term “aqueous suspension” is defined as a suspension comprising at least 50% w/w water.

In one embodiment, the pharmaceutical composition is a freeze-dried composition, whereto the physician or the patient adds solvents and/or diluents prior to use.

In one embodiment, the pharmaceutical composition is a dried composition (e.g. freeze-dried or spray-dried) ready for use without any prior dissolution.

In one embodiment, the invention relates to a pharmaceutical composition comprising an aqueous solution of a modified protein, such as a hGH variant and a buffer, wherein said hGH variant is present in a concentration from 0.1-100 mg/ml or above, and wherein said composition has a pH from about 2.0 to about 10.0.

In one embodiment, the pH of the pharmaceutical composition is selected from the list consisting of 2.0, through 10.0 with an upward gradation of 0.1, for ex. 2.1, 2.2. 2.3 and so on.

In one embodiment, the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In one embodiment, the composition further comprises a pharmaceutically acceptable preservative. In one embodiment, the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In one embodiment, the preservative is present in a concentration from 0.1 mg/ml to 20 mg/ml. In a further embodiment of the invention the preservative is present in a concentration from 0.1 mg/ml to 5 mg/ml. In one embodiment, the preservative is present in a concentration from 5 mg/ml to 10 mg/ml. In one embodiment, the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 20^{th} edition, 2000.

In one embodiment, the composition further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glycans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects obtained using the methods of the invention. In one embodiment, the sugar or sugar alcohol concentration is between about 1 mg/ml and about 150 mg/ml. In one embodiment, the isotonic agent is present in a concentration from 1 mg/ml to 50 mg/ml. In one embodiment, the isotonic agent is present in a concentration from 1 mg/ml to 7 mg/ml. In one embodiment, the isotonic agent is present in a concentration from 8 mg/ml to 24 mg/ml. In one embodiment, the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 20^{th} edition, 2000.

“Growth hormone” or “GH” as used in the current disclosure refers to growth hormone from any species including that of avian, equine, porcine, bovine or ovine, preferably of mammalian origin and more preferably human. Any other polypeptide which exhibits growth hormone-like activity, its fragments and derivatives are included within the meaning of GH as referred in this invention.

The wild type DNA and amino acid sequences of human growth hormone (hGH) have been reported. The amino acid sequence can be seen as SEQ ID No.1. The present invention describes novel hGH variants with N-glycosylation sites introduced by site-specific mutagenesis. The hGH variants of the present invention can be expressed in any recombinant expression system that is capable of glycosylation.

The amino acid substitutions in the hGH variant sequence of the present invention has a notation that defines the hGH variants, for example, the amino acid substitutions are indicated by a letter representing the wild-type residue in single letter code, a number indicating the amino acid position in the wild type sequence, and a second letter indicating the substituted amino acid residue, for example L101S wherein amino acid L at position 101 is replaced by amino acid S. Multiple mutants are indicated by a series of single mutants separated by “+”, for example will L93N+A98N+L101T+G104N designate a mutant carrying all these mutations.

“Plasmid” and “vector” and “plasmid vector” are most commonly used interchangeably which is the case in the current specification. The terms are intended to encompass any nucleic acid construct which, following transfection into a host cell, is capable of replication, either independently of the host genome or by being incorporated into the host genome.

“Expression vector” as meant in the disclosure is an embodiment of a vector and refers to a nucleic acid construct containing a nucleic acid sequence which is operably linked to a suitable control sequence capable of effecting the expression of said nucleic acid in a suitable host. Such control sequences include a promoter to effect transcription, an optional operator sequence to control such transcription, a sequence encoding suitable mRNA ribosome binding sites, and sequences which control termination of transcription and translation. In one embodiment, an expression vector according to the present invention is a eukaryotic expression vector suitable for recombinant expression in a host cell, which host cell is capable of introducing a N-glycolysation at the motif N-X-S/T in a polypeptide comprising such motif. In one embodiment, an expression vector according to the present invention is an expression vector suitable for expression in a CHO cell.“Operably linked” as meant in the disclosure is that they are functionally related to each other within the context of DNA or polypeptide. For example, a presequence is operably linked to a peptide if it functions as a signal sequence, participating in the secretion of the mature form of the protein, most probably involving cleavage of the signal sequence. A promoter is operably linked to a coding sequence if it controls the transcription of the sequence; a ribosome binding site is operably linked to a coding sequence if it is positioned so as to permit translation.

As used herein, an “oligosaccharide chain” refers to the entire oligosaccharide structure that is covalently linked to a single amino acid residue. A “N-glycan” refers to the entire oligosaccharide structure that is covalently linked to a single asparagine residue. An “antenna” refers to a branch of an oligosaccharide chain. N-glycans may be mono-, bi-, tri-, tetra, penta, hexa or hepta-antennary. Each antenna may comprise a sialic acid moiety.

The invention relates to a preparation comprising an N-glycosylated human growth hormone variant, which N-glycosylated human growth hormone variant is a human growth hormone variant as described herein, which human growth hormone variant has been glycosylated with one or more N-glycans, wherein said N-glycan(s) has been attached to one or more of the N-glycosylation motif(s) (N-X-S/T) in said human growth hormone variant, which N-glycosylation motif(s) are not present in the wild type human growth hormone and wherein at least 50% of the N-glycans comprise at least one sialic acid moiety. In one embodiment at least 60% of the N-glycans comprise at least one sialic acid moiety, such as at least 70, 75, 80, 85, 90 or 95% of the N-glycans comprise at least one sialic acid moiety. In case of branched oligosaccharide chains each N-glycan may comprise a large number of sialic acid moieties, such as up to 5, 8, 10, 12, 14 or 16 sialic acid moieties.

Illustrative embodiments according to the invention are described in the following, not to be interpreted as limiting for the scope the invention.

### Embodiments

1. A human growth hormone variant, wherein said variant comprises an amino acid sequence comprising one or more N-glycosylation motifs (N-X-S/T), which are not present in the wild type human growth hormone.
2. A human growth hormone variant according to embodiment 1, wherein at least one of said N-glycosylation motifs (N-X-S/T) not present in the wild type human growth hormone have been generated by introducing a mutation selected from the group consisting of S55N, Q69N, E74S, E74T, R77N, I83N, L93N, A98N, L101S, L101T, G104N, S106N, Y111S, Y111T, I121N, D130N, K140N, T142N, G161S, G161T and E186N.
3. A human growth hormone variant according to embodiment 1, wherein at least one of said N-glycosylation motifs (N-X-S/T) not present in the wild type human growth hormone have been generated by introducing one or more mutation(s)/mutations pair(s) selected from the group consisting of: K41N, Q49N, S55N, E65T, E65T, E65N, Q69N, E74S, E74T, R77N, I83N, L93N, A98N, L101S, L101T, G104N, S106N, Y111S, Y111T, I121N, D130N, P133N, K140N, T142N, G161S, G161T, E186N, R19N+H21S/T, A34N+I36S/T, L45N+N47S/T, I58N+P59F, S62N+R64S/T, S71N+L73S/T, K115N+L117S/T, R127N+E129S/T, L128N+D130S/T and T175N+L177S/T.
4. A human growth hormone variant according to any of the embodiments 1 to 3, wherein at least one of said N-glycosylation motifs (N-X-S/T) not present in the wild type human growth hormone have been generated by introducing one or more mutation(s)/mutations pair(s)selected from the group consisting of: K41N, Q49N, E65T, E65N, Q69N, E74T, R77N, I83N, L93N, A98N, L101T, G104N, S106N, Y111T, I121N, D130N, P133N, K140N, T142N, T148N, G161T, E186N, R19N+H21S, A34N+I36S, L45N+N47S, I58N+P59F, S62N+R64T, S71 N+L73T, K115N+L117T, R127N+E129T, L128N+D130T and T175N+L177S.
5. A human growth hormone variant according to any of the embodiments 1, 3 and 4, wherein all of said N-glycosylation motifs (N-X-S/T) not present in the wild type human growth hormone have been generated by introducing one or more mutation(s)/mutations pair(s) selected from the group consisting of: K41N, Q49N, E65T, E65N, E74T, L93N, A98N, L101T, G104N, Y111T, P133N, K140N, G161T, E186N, R19N+H21S, I58N+P59F, S62N+R64T, S71N+L73T, R127N+E129T and L128N+D130T.
6. A human growth hormone variant according to embodiment 1 or 2, wherein all said N-glycosylation motifs (N-X-S/T) not present in the wild type human growth hormone have been generated by introducing a mutation independently selected from the group consisting of S55N, Q69N, E74S, E74T, R77N, I83N, L93N, A98N, L101S, L101T, G104N, S106N, Y111S, Y111T, I121N, D130N, K140N, T142N, G161S, G161T and E186N.
7. A human growth hormone variant according to any of embodiments 1, 2 and 3, wherein at least one of said N-glycosylation motifs (N-X-S/T) have been generated by introducing a mutation selected from the group consisting of Q69N, R77N, I83N, L93N, A98N, L101T, G104N, S106N, Y111T, I121N, D130N, K140N, G161T, and E186N.
8. A human growth hormone variant according to embodiment 7, wherein all said N-glycosylation motifs (N-X-S/T) have been generated by introducing a mutation independently selected from the group consisting of Q69N, R77N, I83N, L93N, A98N, L101T, G104N, S106N, Y111T, I121N, D130N, K140N, G161T, and E186N.
9. A human growth hormone variant according to embodiment 5, wherein at least one of said N-glycosylation motifs (N-X-S/T) have been generated by introducing one or more mutation(s)/mutations pair(s) selected from the group of: Q49N, E65N, L93N, A98N, L101T G104N, S71N+L73T and R127N+E129T.
10. A human growth hormone variant according to any of embodiments 1 to 9, wherein at least one of said N-glycosylation motifs (N-X-S/T) have been generated by introducing a mutation selected from the group consisting of L93N, A98N, L101T and G104N.
11. A human growth hormone variant according to embodiment 10, wherein all said N-glycosylation motifs (N-X-S/T) have been generated by introducing a mutation independently selected from the group consisting of L93N, A98N, L101T and G104N.
12. A human growth hormone variant according to any of embodiments 1 to 10, wherein at least one of said N-glycosylation motifs (N-X-S/T) have been generated by introducing a mutation selected from the group consisting of L93N, A98N and G104N.
13. A human growth hormone variant according to embodiment 12, wherein all said N-glycosylation motifs (N-X-S/T) have been generated by introducing a mutation independently selected from the group consisting of L93N, A98N and G104N.
14. A human growth hormone variant according to any of embodiments 1 to 10, wherein at least one of said N-glycosylation motifs (N-X-S/T) have been generated by introducing a mutation selected from the group consisting of L93N, L101T and G104N.
15. A human growth hormone variant according to embodiment 14, wherein all said N-glycosylation motifs (N-X-S/T) have been generated by introducing a mutation selected from the group consisting of L93N, L101T and G104N.
16. A human growth hormone variant according to any of embodiments 1 to 15 comprising exactly one N-glycosylation motif (N-X-S/T), which is not present in the wild type human growth hormone.
17. A human growth hormone variant according to any of embodiments 1 to 15 comprising at least two N-glycosylation motifs (N-X-S/T), which are not present in the wild type human growth hormone.
18. A human growth hormone variant according to embodiment 17 comprising exactly two N-glycosylation motifs (N-X-S/T), which are not present in the wild type human growth hormone.
19. A human growth hormone variant according to embodiment 17 comprising at least three N-glycosylation motifs (N-X-S/T), which are not present in the wild type human growth hormone.
20. A human growth hormone variant according to embodiment 19 comprising exactly three N-glycosylation motifs (N-X-S/T), which are not present in the wild type human growth hormone.
21. A human growth hormone variant according to embodiment 20, wherein the three N-glycosylation motifs (N-X-S/T) not present in the wild type human growth hormone has been generated by introduction of the mutations L93N, A98N and G104N.
22. A human growth hormone variant according to embodiment 20, wherein the three N-glycosylation motifs (N-X-S/T) not present in the wild type human growth hormone has been generated by introduction of the mutations L93N, L101T and G104N.
23. A human growth hormone variant according to embodiment 19 comprising at least four N-glycosylation motifs (N-X-S/T), which are not present in the wild type human growth hormone.
24. A human growth hormone variant according to embodiment 23 comprising exactly four N-glycosylation motifs (N-X-S/T), which are not present in the wild type human growth hormone.
25. A human growth hormone variant according to embodiment 24, wherein the four N-glycosylation motifs (N-X-S/T) not present in the wild type human growth hormone has been generated by introduction of the mutations L93N, A98N, L101T and G104N.
26. A human growth hormone variant according to embodiment 23 comprising at least five N-glycosylation motifs (N-X-S/T), which are not present in the wild type human growth hormones.
27. A human growth hormone variant according to embodiment 26 comprising exactly five N-glycosylation motifs (N-X-S/T), which are not present in the wild type human growth hormone.
28. A human growth hormone variant according to embodiment 26 comprising at least six or seven N-glycosylation motifs (N-X-S/T), which are not present in the wild type human growth hormone.
29. A human growth hormone variant according to embodiment 28 comprising exactly six or exactly seven N-glycosylation motifs (N-X-S/T), which are not present in the wild type human growth hormone.
30. A human growth hormone variant according to embodiment 17, wherein N-glycosylation motif(s) (N-X-S/T) not present in the wild type human growth hormone has been generated by introduction of at least two N-glycosylation motif(s) by introduction mutations sets selected from the group of:
a) Q49N and R127N+E129T,
b) Q49N, E65N and G104N,
c) Q49N, L93N and R127N+E129T,
d) Q49N, E65N, L93N and G104N,
e) Q49N, E65N, G104N and R127N+E129T,
f) Q49N, E65N, S71N+L73T, G104N and R127N+E129T,
g) Q49N, E65N, S71N+L73T, L93N, G104N and R127N+E129T,
h) Q49N, E65N, S71N+L73T, L93N, A98N, G104N and R127N+E129T,
i) S71N+L73T, L93N, A98N and G104N,
j) L93N, G104N and R127N+E129T and
k) S71N+L73T, L93N, G104N and R127N+E129T.
   31. A nucleic acid encoding a human growth hormone variant according to any of embodiments 1 to 30.
   32. A nucleic acid according to embodiment 31, which is a DNA construct.
   33. A vector comprising a nucleic acid sequence according to embodiment 31.
   34. A vector according to embodiment 32, which vector is an expression vector.
   35. A vector according to embodiment 33, which vector is an expression vector suitable for recombinant expression in a host cell, which said host cell is capable of introducing a N-glycolysation at the motif N-X-S/T in a polypeptide comprising such motif.
   36. A vector according to embodiment 35, which vector is a eukaryotic expression vector.
   37. A vector according to embodiment 36, which vector is a eukaryotic expression vector suitable for recombinant expression in mammalian cells
   38. A vector according to embodiment 37, which vector is an expression vector suitable for recombinant expression in a CHO cell.
   39. A vector according to any of embodiments 32 to 38, wherein said nucleic acid according to embodiment 31 is a DNA construct.
   40. A host cell comprising a vector according to any of embodiments 22 to 39.
   41. A host cell according to embodiment 40, which cell is capable of performing N-glycolysation at the motif N-X-S/T in a polypeptide comprising such motif.
   42. A host cell according to embodiment 41, which cell is a eukaryotic cell.
   43. A host cell according to embodiment 42, which cell is a mammalian cell.
   44. A host cell according to embodiment 43, which cell is a CHO cell.
   45. A method for preparing an N-glycosylated human growth hormone variant, which method comprises the recombinant expression of a nucleic acid according to embodiment 31 or embodiment 32 in a eukaryotic cell.
   46. A method according to embodiment 45 for preparing an N-glycosylated human growth hormone variant, wherein said nucleic acid is expressed in a mammalian cell.
   47. A method according to embodiment 46 for preparing an N-glycosylated human growth hormone variant, wherein said nucleic acid is expressed in a CHO cell.
   48. A method according to any of embodiments 45 to 47, wherein no further glycosylation or modification of the N-glycans are performed after the recombinant expression of said nucleic acid.
   49. An human growth hormone variant prepared by use of a method according to any of embodiments 45 to 48.
   50. A human growth hormone variant prepared by use of a method according to any of embodiments 45 to 48, wherein the isoelectric point of the said variant is more acidic than the wild-type human growth hormone.
   51. A human growth hormone variant prepared by use of a method according to any of embodiments 45 to 48, wherein said variant changes mobility in an SDS-PAGE, when treated with a glycosidase.
   52. A human growth hormone variant according to embodiment 51, wherein said glycosidase is PNGase F-enzyme or neroaminidase-enzyme.
   53. A human growth hormone variant prepared by use of a method according to any of embodiments 45 to 48, wherein the molecular weight is increased compared to wild-type human growth hormone.
   54. A human growth hormone variant according to any embodiments 49 to 53, wherein the activity of the variant is reduced no more than 100 fold, such as no more than 50, for instance no more than 20, such as no more than 10, for instance no more than 5, such as no more than 2, for instance no more than 1 compared to wild-type human growth hormone.
   55. A human growth hormone variant according to embodiment 54, wherein the activity of the variant is substantially the same as the activity wild-type human growth hormone.
   56. A human growth hormone variant according to any of embodiments 49 to 55, wherein the in vivo circulatory half-life of the human growth hormone variant is prolonged compared to wild-type human growth hormone.
   57. A human growth hormone variant according to any of embodiments 49 to 56, wherein at least 50% of the glycans are sialylated.
   58. An N-glycosylated human growth hormone variant, which variant is N-glycolysated in at least one N-glycosylation motif (N-X-S/T), which motif(s) are not present in the wild type human growth hormone.
   59. An N-glycosylated human growth hormone variant, which N-glycosylated human growth hormone variant is a human growth hormone variant according to any of embodiments 1 to 30 and 49 to 57, which human growth hormone variant has been glycosylated with one or more N-glycans, wherein said N-glycan(s) has been attached to one or more of the N-glycosylation motif(s) (N-X-S/T) in said human growth hormone variant, which N-glycosylation motif(s) are not present in the wild type human growth hormone.
   60. An N-glycosylated human growth hormone variant according to embodiment 59, wherein said human growth hormone variant has been glycosylated with one or more N-glycans, wherein said N-glycan(s) has been attached to all the N-glycosylation motif(s) (N-X-S/T) in said human growth hormone variant, which N-glycosylation motif(s) are not present in the wild type human growth hormone.
   61. An N-glycosylated human growth hormone variant according to embodiment 60, wherein at least one of said N-glycosylation motifs (N-X-S/T) not present in the wild type human growth hormone have been generated by introducing a mutation selected from the group consisting of S55N, Q69N, E74S, E74T, R77N, I83N, L93N, A98N, L101S, L101T, G104N, S106N, Y111S, Y111T, I121N, D130N, K140N, T142N, G161S, G161T and E186N.
   62. An N-glycosylated human growth hormone variant according to embodiment 55, wherein at least one of said N-glycosylation motifs (N-X-S/T) not present in the wild type human growth hormone have been generated by introducing one or more mutation(s)/mutations pair(s) selected from the group consisting of: K41N, Q49N, S55N, E65T, E65N, Q69N, E74S, E74T, R77N, I83N, L93N, A98N, L101S, L101T, G104N, S106N, Y111S, Y111T, I121N, D130N, P133N, K140N, T142N, G161S, G161T, E186N, R19N+H21S, A34N+I36S, L45N+N47S, I58N+P59F, S62N+R64T, S71 N+L73T, K115N+L117T, R127N+E129T, L128N+D130T and T175N+L177S.
   63. An N-glycosylated human growth hormone variant according to embodiment 55, wherein at least one of said N-glycosylation motifs (N-X-S/T) not present in the wild type human growth hormone have been generated by introducing one or more mutation(s)/mutations pair(s) selected from the group consisting of: K41N, Q49N, E65T, E65N, Q69N, E74T, R77N, I83N, L93N, A98N, L101T, G104N, S106N, Y111T, I121N, D130N, P133N, K140N, T142N, T148N, G161T, E186N, R19N+H21S, A34N+I36S, L45N+N47S, I58N+P59F, S62N+R64T, S71N+L73T, K115N+L117T, R127N+E129T, L128N+D130T and T175N+L177S.
   64. An N-glycosylated human growth hormone variant according to embodiment 55, wherein at least one of said N-glycosylation motifs (N-X-S/T) not present in the wild type human growth hormone have been generated by introducing one or more mutation(s)/mutations pair(s) selected from the group consisting of: K41N, Q49N, E65T, E65N, E74T, L93N, A98N, L101T, G104N, Y111T, P133N, K140N, G161T, E186N, R19N+H21S, I58N+P59F, S62N+R64T, S71N+L73T, R127N+E129T and L128N+D130T.
   65. An N-glycosylated human growth hormone variant according to embodiment 61, wherein all said N-glycosylation motifs (N-X-S/T) not present in the wild type human growth hormone have been generated by introducing a mutation independently selected from the group consisting of S55N, Q69N, E74S, E74T, R77N, I83N, L93N, A98N, L101S, L101T, G104N, S106N, Y111S, Y111T, I121N, D130N, K140N, T142N, G161S, G161T and E186N.
   66. An N-glycosylated human growth hormone variant according to embodiment 64, wherein at least one of said N-glycosylation motifs (N-X-S/T) have been generated by introducing a mutation selected from the group consisting of Q69N, R77N, I83N, L93N, A98N, L101T, G104N, S106N, Y111T, I121N, D130N, K140N, G161T, and E186N.
   67. An N-glycosylated human growth hormone variant according to embodiment 64, wherein all said N-glycosylation motifs (N-X-S/T) have been generated by introducing a mutation independently selected from the group consisting of Q69N, R77N, I83N, L93N, A98N, L101T, G104N, S106N, Y111T, I121N, D130N, K140N, G161T, and E186N.
   68. An N-glycosylated human growth hormone variant according to any of embodiments 60 to 67, wherein at least one of said N-glycosylation motifs (N-X-S/T) have been generated by introducing a mutation selected from the group consisting of L93N, A98N, L101T and G104N.
   69. An N-glycosylated human growth hormone variant according to embodiment 67, wherein all said N-glycosylation motifs (N-X-S/T) have been generated by introducing a mutation independently selected from the group consisting of L93N, A98N, L101T and G104N.
   70. An N-glycosylated human growth hormone variant according to any of embodiments 63 to 69, wherein at least one of said N-glycosylation motifs (N-X-S/T) have been generated by introducing a mutation selected from the group consisting of L93N, A98N and G104N.
   71. An N-glycosylated human growth hormone variant according to embodiment 70, wherein all said N-glycosylation motifs (N-X-S/T) have been generated by introducing a mutation independently selected from the group consisting of L93N, A98N and G104N.
   72. An N-glycosylated human growth hormone variant according to any of embodiments 63 to 69, wherein at least one of said N-glycosylation motifs (N-X-S/T) have been generated by introducing a mutation selected from the group consisting of L93N, L101T and G104N.
   73. human growth hormone variant according to embodiment 72, wherein all said N-glycosylation motifs (N-X-S/T) have been generated by introducing a mutation selected from the group consisting of L93N, L101T and G104N.
   74. An N-glycosylated human growth hormone variant according to embodiment 64, wherein at least one of said N-glycosylation motifs (N-X-S/T) have been generated by introducing one or more mutation(s)/mutations pair(s) selected from the group of: Q49N, E65N, L93N, A98N, L101T G104N, S71N+L73T and R127N+E129T.
   75. An N-glycosylated human growth hormone variant according to embodiment 74, wherein N-glycosylation motif(s) (N-X-S/T) not present in the wild type human growth hormone has been generated by introduction of at least two N glycosylation motif(s) by introduction mutations sets selected from the group of:
a) Q49N and R127N+E129T,
b) Q49N, E65N and G104N,
c) Q49N, L93N and R127N+E129T,
d) Q49N, E65N, L93N and G104N,
e) Q49N, E65N, G104N and R127N+E129T,
f) Q49N, E65N, S71N+L73T, G104N and R127N+E129T,
g) Q49N, E65N, S71N+L73T, L93N, G104N and R127N+E129T,
h) Q49N, E65N, S71N+L73T, L93N, A98N, G104N and R127N+E129T,
i) S71N+L73T, L93N, A98N and G104N,
j) L93N, G104N and R127N+E129T and
k) S71N+L73T, L93N, G104N and R127N+E129T.
   76. An N-glycosylated human growth hormone variant according to any of embodiments 49 to 75 comprising exactly one N-glycan.
   77. An N-glycosylated human growth hormone variant according to any of embodiments 49 to 75 comprising at least two N-glycans.
   78. An N-glycosylated human growth hormone variant according to embodiment 77 comprising exactly two N-glycans.
   79. An N-glycosylated human growth hormone variant according to embodiment 77 comprising at least three N-glycans.
   80. An N-glycosylated human growth hormone variant according to embodiment 79 comprising exactly three N-glycans.
   81. An N-glycosylated human growth hormone variant according to embodiment 80, wherein the three N-glycosylation motifs (N-X-S/T) not present in the wild type human growth hormone has been generated by introduction of the mutations L93N, A98N and G104N.
   82. An N-glycosylated human growth hormone variant according to embodiment 80, wherein the three N-glycosylation motifs (N-X-S/T) not present in the wild type human growth hormone has been generated by introduction of the mutations L93N, L101T and G104N.
   83. An N-glycosylated human growth hormone variant according to embodiment 79 comprising at least four N-glycans.
   84. An N-glycosylated human growth hormone variant according to embodiment 83 comprising exactly four N-glycans.
   85. An N-glycosylated human growth hormone variant according to embodiment 84, wherein the four N-glycosylation motifs (N-X-S/T) not present in the wild type human growth hormone has been generated by introduction of the mutations L93N, A98N, L101T and G104N.
   86. An N-glycosylated human growth hormone variant according to embodiment 83 comprising at least five N-glycans.
   87. An N-glycosylated human growth hormone variant according to embodiment 86 comprising exactly five N-glycans.
   88. An N-glycosylated human growth hormone variant according to embodiment 86 comprising at least six N-glycans.
   89. An N-glycosylated human growth hormone variant according to embodiment 88 comprising exactly six N-glycans.
   90. A preparation comprising an N-glycosylated human growth hormone variant, which variant is N-glycolysated in at least one N-glycosylation motif (N-X-S/T), which motif(s) are not present in the wild type human growth hormone.
   91. A preparation according to claim 90, wherein said preparation comprises a human growth hormone variant according to any of embodiments 1 to 30.
   92. A preparation according to claim 90, wherein said preparation comprises an N-glycosylated human growth hormone variant according to any of embodiments 49-89 wherein at least 50% of the N-glycans comprise at least one sialic acid moiety.
   93. A preparation according to any of claims 90 to 92, wherein at least 50% of the N-glycans comprise at least one sialic acid moiety.
   94. A preparation according to embodiment 93, wherein at least 75% of the N-glycans comprise at least one sialic acid moiety.
   95. A preparation according to embodiment 94, wherein at least 90% of the N-glycans comprise at least one sialic acid moiety.
   96. A preparation according to embodiment 95, wherein at least 95% of the N-glycans comprise at least one sialic acid moiety.
   97. A preparation according to any of the embodiments 90-92, wherein at least 20% of said human growth hormone variant is N-glycosylated.
   98. A preparation according to any of the embodiments 90-92, wherein at least 50% of said human growth hormone variant is N-glycosylated.
   99. A preparation according to embodiment 97, wherein at least 50% of said human growth hormone variant is N-glycosylated on all glycosylation motif(s) not present in the wild type human growth hormone.
   100. A method for preparing a pharmaceutical composition comprising a N-glycosylated human growth hormone variant according to any of embodiments 49 to 89, which method comprises the steps of
i) recombinantly expressing a nucleic acid according to embodiment 31 or embodiment 23 in a host cell capable of performing N-glycosylation,
ii) purifying the N-glycosylated human growth hormone variant,
iii) preparing a pharmaceutically acceptable formulation comprising the purified N-glycosylated human growth hormone variant from step ii).
   101. A method for preparing a pharmaceutical composition comprising a N-glycosylated human growth hormone variant according to embodiment 100, wherein said host cell is a eukaryotic cell.
   102. A method according to embodiment 101, which cell is a mammalian cell.
   103. A method according to embodiment 102, which cell is a CHO cell.
   104. A pharmaceutical composition comprising an N-glycosylated human growth hormone variant according to any of embodiments 49 to 89 and a pharmaceutically acceptable carrier.
   105. A pharmaceutical composition comprising a preparation according to any of embodiments 90 to 99 and a pharmaceutically acceptable carrier.
   106. A method of treating a mammal in need of human growth hormone, said method comprising administering to the mammal a therapeutically effective amount of an N-glycosylated human growth hormone variant according to any of embodiments 49 to 89.
   107. A method of treating a mammal in need of human growth hormone, said method comprising administering to the mammal a therapeutically effective amount of a preparation according to any of embodiments 90 to 99.
   The present invention will be further illustrated in the following examples. However, it is to be understood that these examples are for illustrative purposes only, and should not be used to limit the scope of the present invention in any manner.

### EXAMPLES

### Example 1

### Construction of Vectors for Expression of Wild-Type Human Growth Hormone in Mammalian Cells

The nucleotide sequence shown in FIG. 1A was inserted into the plasmid pEE14.4 by means of the Hind III and Eco RI sites flanking the sequence to create the plasmid pGB039. In pGB039, the growth hormone encoding nucleotide sequence was placed under the transcriptional control of the cytomegalo virus (CMV) promoter.

The growth hormone encoding nucleotide sequence in pGB039 was subcloned by insertion between the Hind III and Not I sites of pTT5 to create the plasmid pTVL01.

### Example 2

### Transient Expression of Wild-Type Human Growth Hormone in Mammalian HEK293 Cells

Suspension adapted human embryonal kidney (HEK293F) cells (Freestyle, Invitrogen) were transfected with the pGB039 expression plasmid encoding wild-type human growth hormone per manufacturer's instructions. Briefly, 30 pg of plasmid was incubated 20 min with 40 μl 293fectin (Invitrogen) and added to 3×10⁷ cells in a 125 ml Erlenmeyer flask. The transfected cells were incubated in a shaking incubator (37° C., 8% CO₂ and 125 rpm) for 7 days. Medium samples were harvested daily and analyzed for human growth hormone with an ELISA kit (Roche).

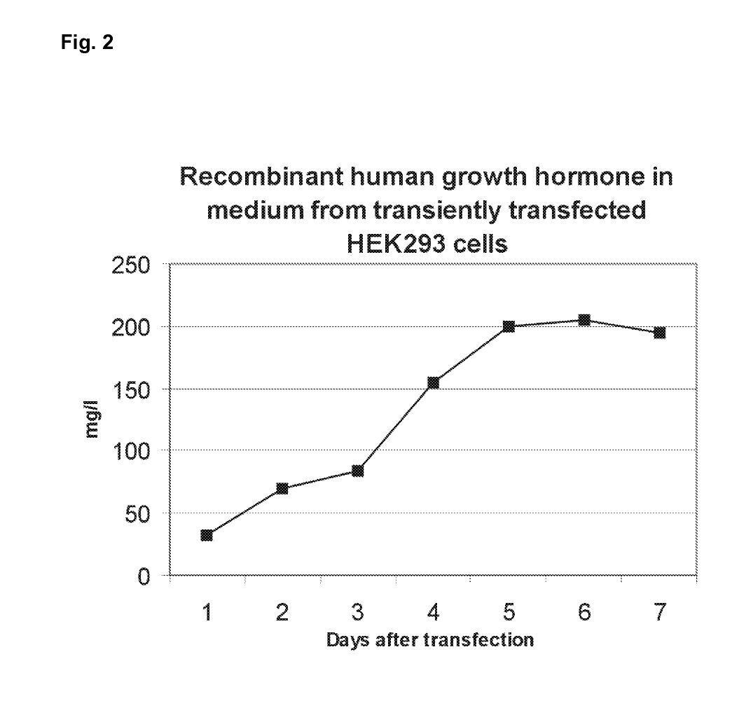

Results of the ELISA are shown in FIG. 2 and demonstrates that the transiently transfected mammalian cells were efficient producers of human growth hormone. Medium harvested 7 days after transfection and dilutions of purified recombinant human growth hormone produced in bacteria was loaded on a SDS-PAGE gel and electrophoresed. The gel was stained with SimpleBlue SafeStain (Invitrogen) and scanned in an Odyssey reader. The medium from transfected cells but not the medium from untransfected cells contained a protein with a molecular weight of approximately 22 kDa that comigrated with recombinant human growth hormone produced in bacteria. This demonstrates that the transiently transfected mammalian cells secreted mature recombinant human growth hormone.

### Example 3

### Identification of Positions in the Human Growth Hormone Protein Suitable for Introduction of N-glycosylation Sites

Amino acid residues on the surface of the human growth hormone protein but not participating in the binding interphase with the growth hormone receptor were considered as most suitable locations for introduction of N-glycosylation sites. Among these residues, amino acids in a sequence context allowing the formation of a potential N-glycosylation site (N-X-S/T) by a single amino acid substitution were selected. However, N-glycosylation sites formed by amino acid substitutions involving cysteine or proline residues were disregarded.

Amino acid positions complying with the above requirements were found by analyzing the file 3hhr from the Protein Data Bank with the Molsoft Browser 3.4-9d (Molsoft) software. The file 3hhr describes the structure of human growth hormone bound to the extracellular domains of two growth hormone receptor molecules and is based on the publication by de Vos et al (1992). This analysis identified the following amino acid substitutions in the amino acid sequence of mature human growth hormone:

S55N, Q69N, E74S, E74T, R77N, I83N, L93N, A98N, L101S, L101T, G104N,

S106N, Y111S, Y111T, I121N, D130N, K140N, T142N, G161S, G161T, and E186N

Each of these sequence alterations introduces a potential N-glycosylation site at a position believed to be on the surface of the protein but not participating in the binding interphase with the growth hormone receptor.

### Example 4

### Generation of Expression Constructs Encoding Human Growth Hormone with One Potential N-glycosylation Site

Constructs encoding human growth hormone variants with potential N-glycosylation sites were generated by site-directed mutagenesis of pTVL01 consisting of pTT5 with an insert encoding wild-type human growth hormone. Constructs encoding variants with one of the mutations Q69N, R77N, I83N, L93N, A98N, L101T, G104N, S106N, Y111T, I121N, K140N, or G161T were generated with the QuikChange Multi Site-Directed Mutagenesis kit (Stratagene) as recommended by the manufacturer using the primers shown in Table 1 (SEQ ID NO 2-13). Constructs encoding variants with one of the mutations D130N or E186N were generated with the QuikChange Site-Directed Mutagenesis kit (Stratagene) as recommended by the manufacturer using the forward primers shown in Table 2 (SEQ ID NO 14-15) and the complementary reverse primers. The sequence of the entire human growth hormone variant encoding nucleotide sequence in the generated constructs was verified by DNA sequencing. The names of the constructs encoding the 14 variants are shown in Table 1 and 2.

TABLE 1
Constructs and the primer for the
mutants of hGH

| Mutation | Mutagenesis primer | Construct |
|---|---|---|
| Q69N | 5′-GCAACAGAGAAGAGACCCAGAATAAGA | pTVL02 |
| | GCAACCTGGAACTGCG-3′ | |
| R77N | 5′-GCAACCTGGAACTGCTGAATATCTCTC | pTVL03 |
| | TGCTGCTGATCC-3′ | |
| I83N | 5′-GGATCTCTCTGCTGCTGAATCAGAGCT | pTVL04 |
| | GGCTGGAAC-3′ | |
| L93N | 5′-CTGGAACCCGTGCAGTTCAATAGAAGC | pTVL05 |
| | GTGTTCGCCAACAG-3′ | |
| A98N | 5′-GTTCCTGAGAAGCGTGTTCAATAACAG | pTVL06 |
| | CCTGGTGTACGGC-3′ | |
| L101T | 5′-GTGTTCGCCAACAGCACGGTGTACGGC | pTVL07 |
| | GCC-3′ | |
| G104N | 5′-CAACAGCCTGGTGTACAACGCCAGCGA | pTVL08 |
| | CAGCAAC-3′ | |
| S106N | 5′-GGTGTACGGCGCCAACGACAGCAACGT | pTVL09 |
| | G-3′ | |
| Y111T | 5′-GCGACAGCAACGTGACCGACCTGCTGA | pTVL10 |
| | AGGAC-3′ | |
| I121N | 5′-CCTGGAAGAAGGCAACCAGACCCTGAT | pTVL11 |
| | GG-3′ | |
| K140N | 5′-CGGCCAGATCTTCAATCAGACCTACAG | pTVL12 |
| | CAAGTTC-3′ | |
| G161T | 5′-GCTCTGCTGAAGAACTACACGCTGCTG | pTVL13 |
| | TACTGCTTCAG-3′ | |

TABLE 2
Constructs and the primer for the
D130N and E186N mutants of hGH

| Mutation | Mutagenesis forward primer | Construct |
|---|---|---|
| D130N | 5′-ATGGGCAGGCTGGAAAATGGCAGCCCC-3′ | pTVL15 |
| E186N | 5′-CAGTGCAGAAGCGTGAATGGGAGCTGCGG | pTVL16 |
| | CTTC-3′ | |

### Example 5

### Transient Expression of Human Growth Hormone with one Potential N-glycosylation Site in Mammalian HEK293 Cells

Suspension adapted human embryonal kidney (HEK293F) cells (Freestyle, Invitrogen) were transfected with the pTVL01 expression plasmid encoding wild-type human growth hormone or the pTVL02-pTFVL16 constructs encoding human growth hormone with potential N-glycosylation sites per manufacturer's instructions. Briefly, 30 μg of each plasmid was incubated 20 min with 40 μl 293fectin (Invitrogen) and added to 3×10⁷ cells in a 125 ml Erlenmeyer flask. The transfected cells were incubated in a shaking incubator (37° C., 8% CO₂ and 125 rpm). Medium harvested 7 days after transfection was incubated 1 h at 37° C. with or without peptide N-glycosidase F (PNGase F), loaded on SDS-PAGE gels and electrophoresed. The gels were stained with SimpleBlue SafeStain (Invitrogen) and scanned in an Odyssey reader. The wild-type growth hormone in the medium from cells transfected with pTVL01 migrated as a band with a molecular weight of approximately 22 kDa and comigrated with recombinant human growth hormone produced in bacteria. The variant growth hormones with potential N-glycosylation sites migrated either as a single band comigrating with wild-type human growth hormone or as two bands, one of which comigrated with wild-type human growth hormone, while the other band had a reduced mobility compared to wild-type human growth hormone (Table 3). Upon incubation with PNGase F, which removes N-glycans, all variants migrated as a single band comigrating with wild-type human growth hormone. Thus, only the N-glycosylation sites at amino acid 93, 98, 99, 104, 109, and 140 of mature human growth hormone were utilized. These six N-glycosylations sites were generated by the mutations L93N, A98N, L101T, G104N, Y111T, and K140N, respectively.

TABLE 3
Utilization of potential N-glycosylation sites in hGH variants

| | Band comigrating | Band with reduced |
|---|---|---|
| | with wild-type hGH | mobility |
| Variant | (unglycosylated hGH) | (glycosylated hGH) |
| Wild-type | 100% | 0% |
| Q69N | 100% | 0% |
| R77N | 100% | 0% |
| I83N | 100% | 0% |
| L93N | <50% | >50% |
| A98N | <50% | >50% |
| L101T | <50% | >50% |
| G104N | <50% | >50% |
| S106N | 100% | 0% |
| Y111T | >75% | <25% |
| I121N | 100% | 0% |
| D130N | 100% | 0% |
| K140N | >75% | <25% |
| G161T | >95% | <5% |
| E186N | >95% | <5% |

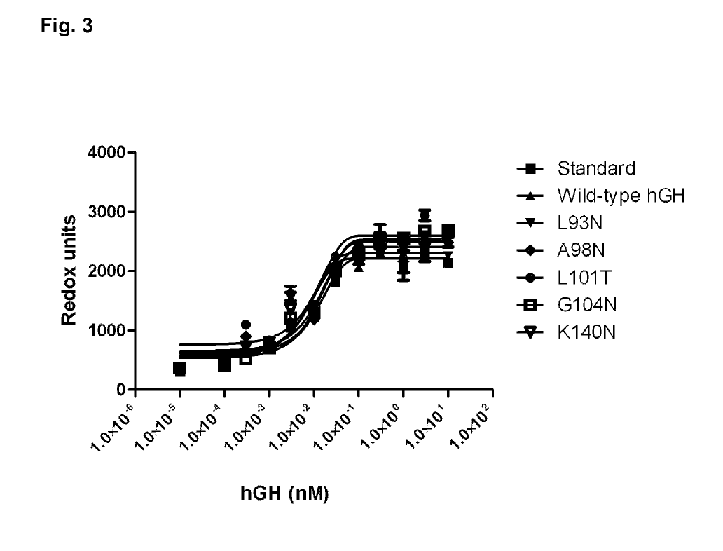

To test the in vitro activity of the human growth hormone mutants with one N-glycosylation site, we examined their proliferation inducing capacity on BAF3-GHR cells. For the growth hormone activity assay, BAF3-GHR cells were incubated for 24 hours at 37° C., 5% CO₂ culture medium without growth hormone (starvation medium). The cells were then seeded in 96 well microtiters plates at a density of 2,22×10⁵ cells/ml in starvation medium. Each well was added 90 μl of the above cell suspension and 10 μl of wildtype or mutant growth hormone in concentrations ranging from 10 nM to 0.1 μM. After seeding, the microtiter plates were incubated for 68 hours at 37° C., 5% CO₂. Next, 30 μl AlamarBlue (Biosource) diluted in starvation medium was added to each well, and the microtiter plates were incubated another 4 hours at 37° C., 5% CO₂. Finally, the microtiter plates were analyzed in a fluorescence plate reader using an excitation filter of 544 nM and an emission filter of 590 nM. AlamarBlue is a redox indicator, which is reduced by reactions innate to cellular metabolism and, therefore, provides an indirect measure of viable cell number, which reflects the growth hormone dependent proliferation of the cells. Results from activity testing of human growth hormone mutants with one N-glycosylation site are shown in FIG. 3.

### Example 6

### Generation of Expression Constructs Encoding Human Growth Hormone with More than One N-glycosylation Site

Constructs encoding human growth hormone variants with 2 or 3 potential N-glycosylation sites were generated by site-directed mutagenesis of pTVL05 consisting of pTT5 with an insert encoding human growth hormone with the mutation L93N with the QuikChange Multi Site-Directed Mutagenesis kit (Stratagene) as recommended by the manufacturer using the primers shown in Table 4. This way, the constructs pTVL05C and pTVL22 were generated. These 2 constructs consist of pTT5 with an insert encoding human growth hormone with the mutations L93N+G104N (pTVL05C) and L93N+L101T+G104N (pTVL22). The A98N mutation was introduced into both these constructs with the QuikChange Site-Directed Mutagenesis kit (Stratagene) as recommended by the manufacturer using the forward primers shown in Table 5 and the complementary reverse primers. This way, the constructs pTVL20 and pTVL21 were generated. These 2 constructs consist of pTT5 with an insert encoding human growth hormone with the mutations L93N+A98N+L101T+G104N (pTVL20) and L93N+A98N+G104N (pTVL21). Thus, the 3 constructs pTVL20, pTVL21, and pTVL22 encode human growth hormone with potential N-glycosylation sites at amino acid 93, 98, 99, and 104 (pTVL20), amino acid 93, 98, and 104 (pTVL21), and amino acid 93, 99 and 104 (pTVL22). The sequence of the entire human growth hormone variant encoding nucleotide sequence in the generated constructs was verified by DNA sequencing.

The growth hormone variant encoding inserts in pTVL20, pTVL21, and pTVL22 were subcloned to pEE14.4 by insertion between the Hind III and Not I sites of pEE14.4. These subclonings gave rise to the constructs pTVL20-SV, pTVL21-SV and pTVL21-SV, respectively.

TABLE 4

| Mutation | Mutagenesis Primer |
|---|---|
| L101T | 5′-GTGTTCGCCAACAGCACGGTGTACGGCGCC-3′ |
| G104N | 5′-CAACAGCCTGGTGTACAACGCCAGCGACAGCAAC-3′ |

TABLE 5

| Mutation | Mutagenesis forward primer |
|---|---|
| TVL05C-A98N | 5′-GTTCAATAGAAGCGTGTTCAACAACAGCACGG |
| | TGTACAAC-3′ |
| TVL22-A98N | 5′-GTTCAATAGAAGCGTGTTCAACAACAGCCTGG |
| | TGTACAAC-3′ |

### Example 7

### Transient Expression of Human Growth Hormone with More than One N-glycosylation Site in Mammalian HEK293 Cells

Suspension adapted human embryonal kidney (HEK293F) cells (Freestyle, Invitrogen) were transfected with the pTVL01 expression plasmid encoding wild-type human growth hormone, pTVL20 encoding human growth hormone with the mutations L93N+A98N+L101T+G104N, pTVL21 encoding human growth hormone with the mutations L93N+-A98N+G104N, or pTVL22 encoding human growth hormone with the mutations L93N+L101T+G104N per manufacturer's instructions. Briefly, 30 μg of each plasmid was incubated 20 min with 40 μl 293fectin (Invitrogen) and added to 3×10⁷ cells in a 125 ml Erlenmeyer flask. The transfected cells were incubated in a shaking incubator (37° C., 8% CO₂ and 125 rpm). Medium harvested 7 days after transfection was incubated 1 h at 37° C. with or without peptide N-glycosidase F (PNGase F), loaded on a SDS-PAGE gel and electrophoresed. The gel was stained with SimpleBlue SafeStain (Invitrogen) and scanned in an Odyssey reader. The variant growth hormones with 3 or 4 potential N-glycosylation sites all migrated as three major bands representing growth hormone with 0, 2 or 3 N-glycans, respectively. Upon incubation with PNGase F, which removes N-glycans, all 3 variants migrated as a single band comigrating with unglycosylated growth hormone. Thus, 3 N-glycosylation sites were utilized in all 3 variants.

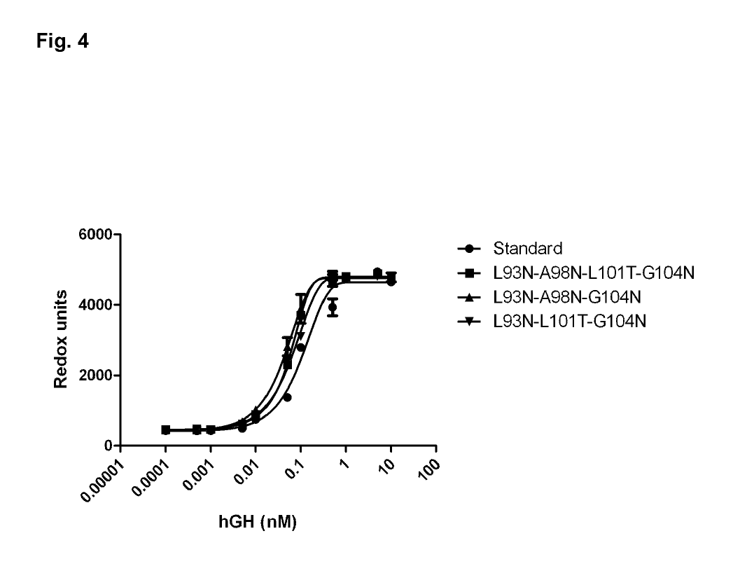

The in vitro activity of the three human growth hormone mutants with more than one N-glycosylation site was examined with BAF3-GHR cell assay described in Example 5 Results from the activity testing are shown in FIG. 4.

### Example 8

### Generation of Stable CHO Cell Lines Producing Human Growth Hormone with More than One Potential N-glycosylation Site

The plasmid pTVL20-SV was electroporated into CHO-K1-SV cells. pTVL20-SV was described in Example 6 and consists of pEE14.4 with an insert encoding human growth hormone with the mutations L93N+A98N+L101T+G104N. Briefly, 1×10⁷ CHO-K1-SV cells were electroporated with 40 μg pTVL20-SV cells and seeded in the wells of 40 microtiter tissue culture plates with medium containing 10% fetal calf serum. The day after transfection, MSX to a final concentration of 50 μM was added to all wells. Cell growth was detected 3-6 weeks post-transfection and growing cells were transferred to 24-well tissue culture plates. As the cells in the 24-well plates reached approximately semi-confluency, they were allowed to grow for 7 days and a standard ELISA procedure on harvested cell culture supernatants were done to select the best yielding cell lines. These cell lines were adapted to growth in serum-free free cell culture medium in shaker flasks, and the best producer cells were identified based on their ability to produce high levels of human growth hormone at high cell densities in an 11 day non-supplemented serum-free culture performed. Selection of the best producer cell lines was based on ELISA, HPLC, and SDS-PAGE on cell culture supernatants.

### Example 9

### Purification of Human Growth Hormone with More than One N-glycosylation Site from Mammalian Cell Culture Supernatant

A CHO-K1-SV cell line generated as described in Example 8 and seeded in a bioreactor was used for production of human growth hormone with the mutations 93N+A98N+L101T+G104N. Medium harvested from the fermentor was cell-depleted and afterwards diluted 10-fold in buffer with a final concentration of 20 mM Triethanolacetate, pH 8.5 at room temperature. The diluted material was loaded onto a 170 ml (ø=5.0 cm, I=8.7 cm) Q Sepharose HP (24-44 μm) anion exchange column (GE Healthcare) in a process driven by an ÄKTA MiniPilot equipment (GE Healthcare). Elution of the material from the column was done with 20 mM Triethanolacetate and 400 mM NaCl, pH 8.5 at room temperature increasing in concentration from 0 to 100% over 14 column volumes (2390 mL). The throughput was registered using UV-absorbance at 254 nm and 280 nm and was collected in fractions. The fractions containing growth hormone were collected in pools.

### Example 10

### Comparison of the Pharmacokinetic Properties of Human Growth Hormone with More than One N-glycosylation Site with Those of Wild-Type Human Growth Hormone

Recombinant wild-type human growth hormone and human growth hormone with the mutations L93N+A98N+L101T+G104N (TVL20) were diluted in buffer consisting of 20 mg/ml glycine, 2 mg/ml mannitol, 2.4 mg/ml NaHCO3, pH adjusted to 8.2 to a final concentration of 150 nmol/ml. 0.1 ml corresponding to 15 nmol of each batch and each compound were administered intravenously via a tail vein (IV) nine male Sprague Dawley rats each. The Sprague Dawley rats were weighing approximately 200-250 g.

For all rats, blood samples were drawn 5 minutes and 1, 2, 4, 8, 18, 24, 48 and 72 hours after dosing. 0.2 ml blood samples were drawn as tail vein puncture using a 23G needle. Blood samples were collected in test tubes containing 8 mM EDTA. Blood samples were kept on ice for a maximum of 20 minutes before centrifugation (1500×g, 4° C., 10 min.). 120 μl plasma was collected from each blood sample, transferred to a test tube and placed on dry ice. Frozen plasma samples were stored at −20° C. until analysis for the content of human growth hormone antigen using compound specific standard curves.

Human growth hormone analogue concentrations were determined by Luminescence Oxygen Channelling Immunoassay (LOCI), which is a homogenous bead based assay. LOCI reagents include two latex bead reagents and biotinyl-mAb 20GS10, which is one part of the sandwich. One of the bead reagents is a generic reagent (donor beads) and is coated with streptavidin and contains a photosensitive dye. The second bead reagent (acceptor beads) is coated with an antibody making up the sandwich. During the assay, the three reactants combine with analyte to form a bead-aggregate-immune complex. Illumination of the complex releases singlet oxygen from the donor beads which channels into the acceptor beads and triggers chemiluminescence which is measured in the EnVision plate reader. The amount of light generated is proportional to the concentration of hGH derivative. 2 μL 40× in LOCI buffer diluted sample/calibrator/control is applied in 384-well LOCI plates. 15 μL of a mixture of biotinyl-mAb 20GS10 and mAb 10G05/M94169 anti-(hGH) conjugated acceptor-beads is added to each well (21-22° C.). The plates are incubated for 1 h at 21-22° C. 30 μL streptavidin coated donor-beads (67 μg/mL) is added to each well and all is incubated for 30 minutes at 21-22° C. The plates are read in an Envision plate reader at 21-22° C. with a filter having a bandwidth of 520-645 nm after excitation by a 680 nm laser. The total measurement time per well is 210 ms including a 70 ms excitation time. The limit of detection for the N-glycosylated human growth hormone analogues were 199, 80 and 350 pM respectively.

Plasma concentration-time data were analysed by non-compartmental pharmacokinetic analysis using WinNonlin Professional (Pharsight Corporation). Calculations were performed using mean concentration-time values from two animals at each time point.

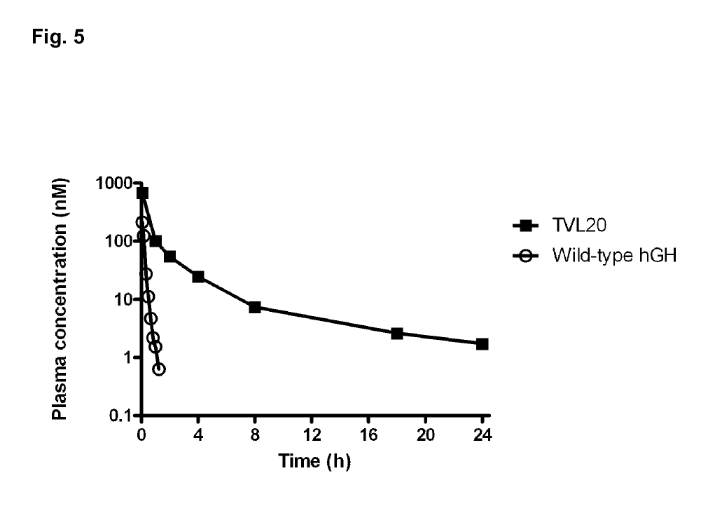

The mean growth hormone antigen concentrations versus time after intravenous dosing are shown in FIG. 5. The estimated pharmacokinetic parameters after intravenous administration are listed in Table 6.

The pharmacokinetic data of human growth hormone with the mutations L93N+A98N+L101T+G104N (TVL20) showed increased exposure in terms of dose corrected area under the plasma concentration-time curve (AUC), reduced clearance and increased plasma in vivo half-life compared to wild-type human growth hormone in Sprague Dawley rats.

TABLE 6
Pharmacokinetic parameters in intravenously dosed Sprague Dawley rats

| | AUC/ | Terminal | | Mean |
|---|---|---|---|---|
| | Dose | half-life | Clearance | Residence |
| Compound | (h/L) | (h) | (L/h) | Time (h) |
| Wildtype human growth | 4.23 | 0.23 | 0.237 | 0.15 |
| hormone | | | | |
| L93N + A98N + L101T + | 41.7 | 7.5 | 0.0240 | 3.6 |
| G104N variant (TVL20) | | | | |

### Example 11

### Identification of Positions in the Human Growth Hormone Protein Suitable for Introduction of N-glycosylation Sites

In a second round of mutations of amino acids, residues on the surface of the human growth hormone protein but not participating in the binding interphase with the growth hormone receptor were considered as most suitable locations for introduction of N-glycosylation sites. Among these residues, amino acids in a sequence context allowing the formation of a potential N-glycosylation site (N-X-S/T) by a single amino acid substitution were preferred. However, amino acids in a sequence context allowing the formation of a potential N-glycosylation site (N-X-S/T) by a double amino acid substitution were also included.

Amino acid positions complying with the above requirements were found by analyzing the files 3hhr and 1hwg from the Protein Data Bank with the Molsoft Browser 3.4-9d (Molsoft) software. The file 3hhr describes the structure of human growth hormone bound to the extracellular domains of two growth hormone receptor molecules and is based on the publication by de Vos et al (1992) and the file 1hwg describes the structure of an antagonist mutant, G120R, of human growth hormone bound to the extracellular domains of two growth hormone receptor molecules and is based on the publication by Sundström et al (1996). This analysis identified the following single amino acid substitutions in the amino acid sequence of mature human growth hormone:

K41N, Q49N, E65S/T, E65N, E74T, P133N, T142N, and T148N,

And the following double amino acid substitutions in the amino acid sequence of mature human growth hormone:

R19N+H21S/T, A34N+I36S, L45N+N47S/T, I58N+P59F, S62N+R64S/T, S71N+L73S/T, K115N+L117S/T, R127N+E129S/T, L128N+D130S/T, and T175N+L177S/T.

Each of these sequence alterations introduces a potential N-glycosylation site at a position believed to be on the surface of the protein but not participating in the binding interphase with the growth hormone receptor.

### Example 12

### Generation of Expression Constructs Encoding Human Growth Hormone with One Potential N-glycosylation Site

Constructs encoding human growth hormone variants with potential N-glycosylation sites were generated by site-directed mutagenesis of pTVL01 consisting of pTT5 with an insert encoding wild-type human growth hormone. Constructs encoding variants with one of the mutation/mutation pairs K41N, Q49N, E65T, E65N, E74T, P133N, T142N, T148N, R19N+H215, A34N+136S, L45N+N47S, I58N+P59F, S62N+R64T, S71N+L73T, K115N+L117T, R127N+E129T, L128N+D130T and T175N+L177S were generated with the QuikChange Site-Directed Mutagenesis kit (Stratagene) as recommended by the manufacturer using the forward primers shown in Table 7 (SEQ ID NO 20-27) and 8 SEQ ID NO 28-37) and the complementary reverse primers. The sequence of the entire human growth hormone variant encoding nucleotide sequence in the generated constructs was verified by DNA sequencing. The names of the constructs encoding the 8 novel variants with a single introduced mutation are shown in Table 7 and the names of the constructs encoding the 10 novel variants with double mutations introduced are shown in Table 8

TABLE 7
Constructs and the primer for mutants
of hGH harboring a single mutation

| Mutation | Mutagenesis primer | Construct |
|---|---|---|
| K41N | 5′-GCCTACATCCCCAAAGAACAG | pTVL40 |
| | AATTACAGCTTTCTGC-3′ | |
| Q49N | 5′-GCTTTCTGCAGAACCCCAATA | pTVL41 |
| | CCTCCCTGTGCTTCAG-3′ | |
| E65T | 5′-CCACCCCCAGCAACAGAACGG | pTVL42 |
| | AGACCCAGCAGAAGAG-3′ | |
| E65N | 5′-CACCCCCAGCAACAGAAATGA | pTVL43 |
| | GACCCAGCAGAAGA-3′ | |
| E74T | 5′-CCAGCAGAAGAGCAACCTGAC | pTVL44 |
| | GCTGCTGAGGATCTCTCTGC-3′ | |
| P133N | 5′-CTGGAAGATGGCAGCAACAGG | pTVL45 |
| | ACCGGCCAGAT-3′ | |
| T142N | 5′-CCAGATCTTCAAGCAGAACTA | pTVL46 |
| | CAGCAAGTTCGACA-3′ | |
| T148N | 5′-CTACAGCAAGTTCGACAACAA | pTVL47 |
| | CAGCCACAACGACG-3′ | |

TABLE 8
Constructs and the primer for mutants of hGH harboring double mutations

| Mutations | Mutagenesis primer | Construct |
|---|---|---|
| R19N + H21S | 5′-GCCATGCTGAGGGCCCACAATCTGAGCCAGCTGGCCTTTG-3′ | pTVL50 |
| A34N + I36S | 5′-CCTTTGACACCTACCAGGAATTTGAGGAAAACTACAGCCCCAAAGAACAGAA-3′ | pTVL51 |
| L45N + N47S | 5′-ATCCCCAAAGAACAGAAGTACAGCTTTAATCAGAGCCCCCAGACCTCCC-3′ | pTVL52 |
| I58N + P59F | 5′-GTGCTTCAGCGAGAGCAACTTCACCCCCAGCAACAGAG-3′ | pTVL53 |
| S62N + R64T | 5′-GAAGAGACCCAGCAGAAGAACAACACGGAACTGCTGAGGATC-3′ | pTVL54 |
| S71N + L73T | 5′-GAAGAGACCCAGCAGAAGAACAACACGGAACTGCTGAGGATC-3′ | pTVL55 |
| K115N + L117T | 5′-ACGTGTACGACCTGCTGAATGACAATGAAGAAGGCATCCAGACCC-3′ | pTVL56 |
| R127N + E129T | 5′-TCCAGACCCTGATGGGCAATCTGACGGATGGCAGCCCCAGGACC-3′ | pTVL57 |
| L128N + D130T | 5′-CAGACCCTGATGGGCAGGAATGAAACTGGCAGCCCCAGGACCGG-3′ | pTVL58 |
| T175N + L177S | 5′-CATGGACAAGGTGGAGAACTTCTCGAGGATCGTGCAGTGCA-3′ | pTVL59 |

### Example 13

### Transient Expression of Human Growth Hormone with One Potential N-glycosylation Site in Mammalian HEK293 Cells

Suspension adapted human embryonal kidney (HEK293F) cells (Freestyle, Invitrogen) were transfected with the pTVL01 expression plasmid encoding wild-type human growth hormone or the pTVL40-pTVL59 constructs encoding human growth hormone with potential N-glycosylation sites per manufacturer's instructions. Briefly, 30 pg of each plasmid was incubated 20 min with 40 μl 293fectin (Invitrogen) and added to 3×10⁷ cells in a 125 ml Erlenmeyer flask. The transfected cells were incubated in a shaking incubator (37° C., 8% CO₂ and 125 rpm). Medium harvested 7 days after transfection was incubated 1 h at 37° C. with or without peptide N-glycosidase F (PNGase F), loaded on SDS-PAGE gels and electrophoresed. The gels were stained with SimpleBlue SafeStain (Invitrogen) and scanned in an Odyssey reader. The wild-type growth hormone in the medium from cells transfected with pTVL01 migrated as a band with a molecular weight of approximately 22 kDa and comigrated with recombinant human growth hormone produced in bacteria. The variant growth hormones with potential N-glycosylation sites migrated either as a single band comigrating with wild-type human growth hormone or as two bands, one of which comigrated with wild-type human growth hormone, while the other band had a reduced mobility compared to wild-type human growth hormone (Table 9 and 10). Upon incubation with PNGase F, which removes N-glycans, all variants migrated as a single band comigrating with wild-type human growth hormone. The band with reduced mobility represents N-glycosylated growth hormone. Thus, only the N-glycosylation sites at amino acid 41, 49, 63, 65, 72, 133, 19, 58, 62, 71, 127, and 128 of mature human growth hormone were utilized. These 12 N-glycosylations sites were generated by the mutations K41N, Q49N, E65T, E65N, E74T, P133N, R19N+H21S, I58N+P59F, S62N+R64T, S71N+L73T, R127N+E129T, and L128N+D130T, respectively.

TABLE 9
Utilization of potential N-glycosylation sites in hGH variants
with single mutations

| | Band comigrating | |
|---|---|---|
| | with wild-type hGH | Band with reduced mobility |
| Variant | (unglycosylated hGH) | (glycosylated hGH) |
| K41N | >75% | <25% |
| Q49N | <50% | >50% |
| E65T | <50% | >50% |
| E65N | <50% | >50% |
| E74T | >75% | <25% |
| P133N | >75% | <25% |
| T142N | 100% | 0% |
| T148N | 100% | 0% |

TABLE 10
Utilization of potential N-glycosylation sites in hGH variants
with double mutations

| | Band comigrating | Band with reduced |
|---|---|---|
| | with wild-type hGH | mobility |
| Variant | (unglycosylated hGH) | (glycosylated hGH) |
| R19N + H21S | <50% | >50% |
| A34N + I36S | 100% | 0% |
| L45N + N47S | 100% | 0% |
| I58N + P59F | >75% | <25% |
| S62N + R64T | <50% | >50% |
| S71N + L73T | <50% | >50% |
| K115N + L117T | 100% | 0% |
| R127N + E129T | <50% | >50% |
| L128N + D130T | <50% | >50% |
| T175N + L177S | 100% | 0% |

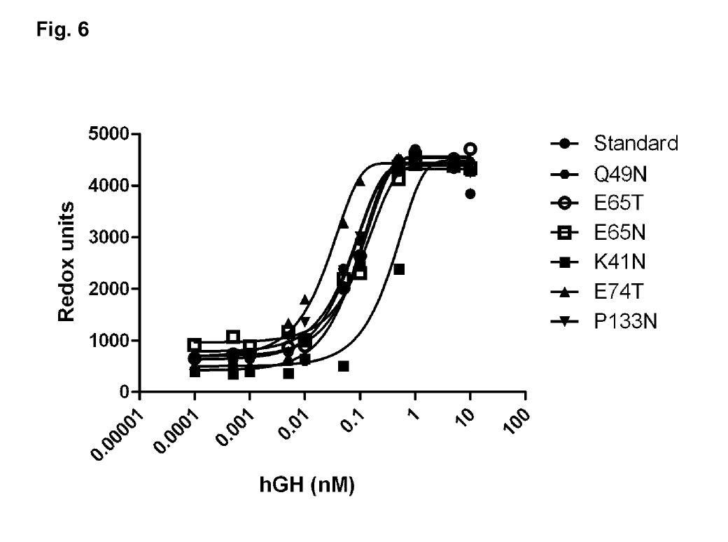

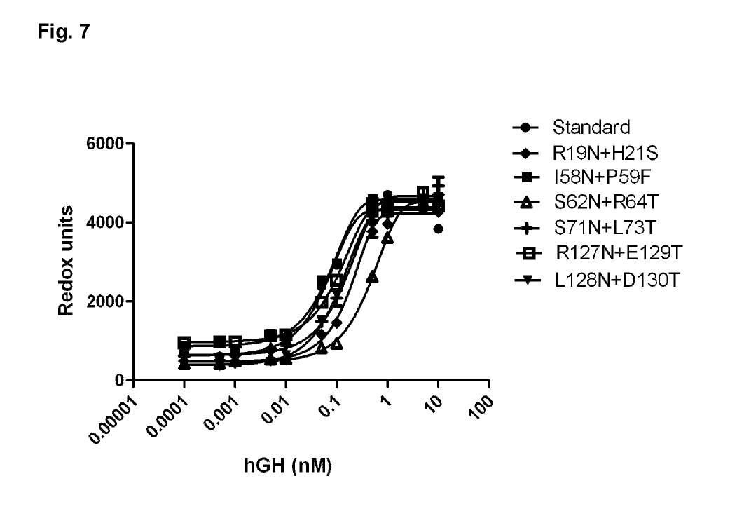

To test the in vitro activity of the 12 human growth hormone mutants with one N-glycosylation site, we examined their proliferation inducing capacity on BAF3-GHR cells. For the growth hormone activity assay, BAF3-GHR cells were incubated for 24 hours at 37° C., 5% CO₂ culture medium without growth hormone (starvation medium). The cells were then seeded in 96 well microtiters plates at a density of 2,22×10⁵ cells/ml in starvation medium. Each well was added 90 μl of the above cell suspension and 10 μl of wildtype or mutant growth hormone in concentrations ranging from 10 nM to 0.1 ρM. After seeding, the microtiter plates were incubated for 68 hours at 37° C., 5% CO₂. Next, 30 μl AlamarBlue (Biosource) diluted in starvation medium was added to each well, and the microtiter plates were incubated another 4 hours at 37° C., 5% CO₂. Finally, the microtiter plates were analyzed in a fluorescence plate reader using an excitation filter of 544 nM and an emission filter of 590 nM. AlamarBlue is a redox indicator, which is reduced by reactions innate to cellular metabolism and, therefore, provides an indirect measure of viable cell number, which reflects the growth hormone dependent proliferation of the cells. Results from activity testing of the human growth hormone mutants with one or more N-glycosylation site are shown in FIG. 6 and FIG. 7.

### Example 14

### Generation of Expression Constructs Encoding Human Growth Hormone with More than One N-glycosylation Site

Constructs encoding human growth hormone variants with 2, 3, 4, 5, 6, or 7 potential N-glycosylation sites were generated by polymerase chain reaction (PCR) with 20-mer forward and reverse primers and 40-mer oligonucleotides covering the entire human growth hormone encoding cDNA. Restriction sites of enzymes Pme I and Eco RI were introduced in front of the human growth hormone encoding cDNA and restriction sites of enzymes Hind III, Not I and Nae I were introduced following the human growth hormone encoding cDNA. Table 11 shows the 20-mer primer (SEQ ID NO 38) and 40-mer oligonucleotides (SEQ ID NO 39-56) used to build the forward strand of the wild-type human growth hormone cDNA.

A 20-mer primer and eighteen 40-mer oligonucleotides that described the complementary strand in a fashion of 20 by overlaps with the corresponding forward strands (i.e. overlapping with the forward primer and the 20 first bases of hGH oligonucleotide 1 or with the 20 last bases of hGH oligonucleotide 1 and the 20 first bases of hGH oligonucleotide 2), were also included in the PCRs.

To introduce mutations to the wild-type human growth hormone cDNA, a given hGH 40-mer oligonucleotide were replaced with a 40-mer oligonucleotide harbouring the mutation(s) of choice. The complementary oligonucleotide(s) were exchanged in a similar way. In Table 12, the 40-mer oligonucleotides used to introduce the mutations to the forward strand are presented. The oligonucleotides are named with the constructs describing the relevant mutation.

This way, 11 different PCR products with mutated human growth hormone cDNA were generated. By digestion of the PCR products and the pTT5 vector with restriction enzymes Hind III and Eco RI and standard ligation procedures, the PCR products were inserted into pTT5. This way, the constructs pTVL60, pTVL61, pTVL62, pTVL63, pTVL64, pTVL66, pTVL67, pTVL68, pTVL70, pTVL71, and pTVL72 were generated. These 11 constructs consist of pTT5 with an insert encoding human growth hormone with the mutations Q49N+R127N+E129T (pTVL60), Q49N+E65N+G104N (pTVL61), Q49N+L93N+R127N+E129T (pTVL62), Q49N+E65N+L93N+G104N (pTVL63), Q49N+E65N+G104N+R127N+E129T (pTVL64), Q49N+E65N+S71N+L73T+G104N+R127N+E129T (pTVL66), Q49N+E65N+S71N+L73T+L93N+G104N+R127N+E129T (pTVL67), Q49N+E65N+S71N+L73T+L93N+A98N+G104N+R127N+E129T (pTVL68), S71N+L73T+L93N+A98N+G104N (pTVL70), L93N+A98N+G104N+R127N+E129T (pTVL71), and S71N+L73T+L93N+A98N+G104N+R127N+E129T (pTVL72).

Thus, the 11 constructs pTVL60, pTVL61, pTVL62, pTVL63, pTVL64, pTVL66, pTVL67, pTVL68, pTVL70, pTVL71, and pTVL72 encode human growth hormone with potential N-glycosylation sites at amino acid 49 and 127 (pTVL60), amino acid 49, 65, and 104 (pTVL61), amino acid 49, 93, and 127 (pTVL62), amino acid 49, 65, 93, and 104 (pTVL63), amino acid 49, 65, 104, and 127 (pTVL64), amino acid 49, 65, 71, 104, and 127 (pTVL66), amino acid 49, 65, 71, 93, 104, and 127 (pTVL67), amino acid 49, 65, 71, 93, 98, 104, and 127 (pTVL68), amino acid 71, 93, 98, and 104 (pTVL70), amino acid 93, 98, 104, and 127 (pTVL71,) and amino acid 71, 93, 98, 104, and 127 (pTVL72). The sequences of the entire human growth hormone variant encoding cDNAs in the generated constructs were verified by DNA sequencing.

TABLE 11

| DNA oligonucleotide | Sequence |
|---|---|
| hGH forward primer | 5′-CAAGTTTAAACGGATCTCTA-3′ |
| hGH oligonucleotide 1 | 5′-GCGAATTCCCTGCAATGGCCACCGGCAGCAGGACCAGCCT-3′ |
| hGH oligonucleotide 2 | 5′-GCTGCTGGCCTTCGGCCTGCTGTGCCTGCCCTGGCTGCAG-3′ |
| hGH oligonucleotide 3 | 5′-GAAGGATCCGCCTTTCCAACCATCCCCCTGAGCAGGCTGT-3′ |
| hGH oligonucleotide 4 | 5′-TCGACAACGCCATGCTGAGGGCCCACAGGCTGCACCAGCT-3′ |
| hGH oligonucleotide 5 | 5′-GGCCTTTGACACCTACCAGGAATTTGAGGAAGCCTACATC-3′ |
| hGH oligonucleotide 6 | 5′-CCCAAAGAACAGAAGTACAGCTTTCTGCAGAACCCCCAGA-3′ |
| hGH oligonucleotide 7 | 5′-CCTCCCTGTGCTTCAGCGAGAGCATCCCCACCCCCAGCAA-3′ |
| hGH oligonucleotide 8 | 5′-CAGAGAAGAGACCCAGCAGAAGAGCAACCTGGAACTGCTG-3′ |
| hGH oligonucleotide 9 | 5′-AGGATCTCTCTGCTGCTGATCCAGAGCTGGCTGGAACCCG-3′ |
| hGH oligonucleotide 10 | 5′-TGCAGTTCCTGAGAAGCGTGTTCGCCAACAGCCTGGTGTA-3′ |
| hGH oligonucleotide 11 | 5′-CGGCGCCAGCGACAGCAACGTGTACGACCTGCTGAAGGAC-3′ |
| hGH oligonucleotide 12 | 5′-CTGGAAGAAGGCATCCAGACCCTGATGGGCAGGCTGGAAG-3′ |
| hGH oligonucleotide 13 | 5′-ATGGCAGCCCCAGGACCGGCCAGATCTTCAAGCAGACCTA-3′ |
| hGH oligonucleotide 14 | 5′-CAGCAAGTTCGACACCAACAGCCACAACGACGACGCTCTG-3′ |
| hGH oligonucleotide 15 | 5′-CTGAAGAACTACGGGCTGCTGTACTGCTTCAGAAAGGACA-3′ |
| hGH oligonucleotide 16 | 5′-TGGACAAGGTGGAGACCTTCCTGAGGATCGTGCAGTGCAG-3′ |
| hGH oligonucleotide 17 | 5′-AAGCGTGGAGGGGAGCTGCGGCTTCTAGTAGCAAGCTTGC-3′ |
| hGH oligonucleotide 18 | 5′-TAGCGGCCGCTCGAGGCCGGCAAGGCCGGATCCCCCGACC-3′ |

TABLE 12
Primers for mutants of hGH harboring one or more mutations

| DNA oligonucleotide | Sequence | Mutation(s) |
|---|---|---|
| hGH oligonucleotide 6 TVL41 | 5′-CCCAAAGAACAGAAGTACAG | Q49N |
| | CTTTCTGCAGAACCCCAATA-3′ | |
| hGH oligonucleotide 8 TVL43 | 5′-CAGAAATGAGACCCAGCAGA | E65N |
| | AGAGCAACCTGGAACTGCTG-3′ | |
| hGH oligonucleotide 8 TVL55 | 5′-CAGAGAAGAGACCCAGCAGA | S71N + L73T |
| | AGAACAACACGGAACTGCTG-3′ | |
| hGH oligonucleotide 8 | 5′-CAGAAATGAGACCCAGCAGA | E65N + S71N + L73T |
| TVL43 + TVL55 | AGAACAACACGGAACTGCTG-3′ | |
| hGH oligonucleotide 10 TVL05 | 5′-TGCAGTTCAATAGAAGCGTG | L93N |
| | TTCGCCAACAGCCTGGTGTA-3′ | |
| hGH oligonucleotide 10 | 5′-TGCAGTTCAATAGAAGCGTG | L93N + A98N |
| TVL05 + pTVL06 | TTCAATAACAGCCTGGTGTA-3′ | |
| hGH oligonucleotide 11 TVL08 | 5′-CAACGCCAGCGACAGCAACG | G104N |
| | TGTACGACCTGCTGAAGGAC-3′ | |
| hGH oligonucleotide 12 TVL57 | 5′-CTGGAAGAAGGCATCCAGAC | R127N + E129T |
| | CCTGATGGGCAATCTGACGG-3′ | |

### Example 15

### Transient Expression of Human Growth Hormone with More than One N-glycosylation Site in Mammalian HEK293 Cells

Suspension adapted human embryonal kidney (HEK293F) cells (Freestyle, Invitrogen) were transfected with the pTVL01 expression plasmid encoding wild-type human growth hormone, pTVL60 encoding human growth hormone with the mutations Q49N+R127N+E129T, pTVL61 encoding human growth hormone with the mutations Q49N+E65N+G104N, pTVL62 encoding human growth hormone with the mutations Q49N+L93N+R127N+E129T, pTVL63 encoding human growth hormone with the mutations Q49N+E65N+L93N+G104N, pTVL64 encoding human growth hormone with the mutations Q49N+E65N+G104N+R127N+E129T, pTVL66 encoding human growth hormone with the mutations Q49N+E65N+S71N+L73T+G104N+R127N+E129T, pTVL67 encoding human growth hormone with the mutations Q49N+E65N+S71N+L73T+L93N+G104N+R127N+E129T, pTVL68 encoding human growth hormone with the mutations Q49N+E65N+S71N+L73T+L93N+A98N+G104N+R127N+E129T, pTVL70 encoding human growth hormone with the mutations S71N+L73T+L93N+A98N+G104N, pTVL71 encoding human growth hormone with the mutations L93N+A98N+G104N+R127N+E129T, and pTVL72 encoding human growth hormone with the mutations S71N+L73T+L93N+A98N+G104N+R127N+E129T per manufacturer's instructions. Briefly, 30 μg of each plasmid was incubated 20 min with 40 μl 293fectin (Invitrogen) and added to 3×10⁷ cells in a 125 ml Erlenmeyer flask. The transfected cells were incubated in a shaking incubator (37° C., 8% CO₂ and 125 rpm). Medium harvested 7 days after transfection was incubated 1 h at 37° C. with or without peptide N-glycosidase F (PNGase F), loaded on a SDS-PAGE gel and electrophoresed. The gel was stained with SimpleBlue SafeStain (Invitrogen) and scanned in an Odyssey reader. The variant growth hormones with 2-7 potential N-glycosylation sites all migrated as major bands representing growth hormone with the maximum number of glycans and to a minor extent as species presenting 0 to 6 (where possible) glycans. Upon incubation with PNGase F, which removes N-glycans, all variants migrated as a single band comigrating with unglycosylated growth hormone. Thus, the maximum numbers of N-glycosylation sites were utilized in all 11 variants.

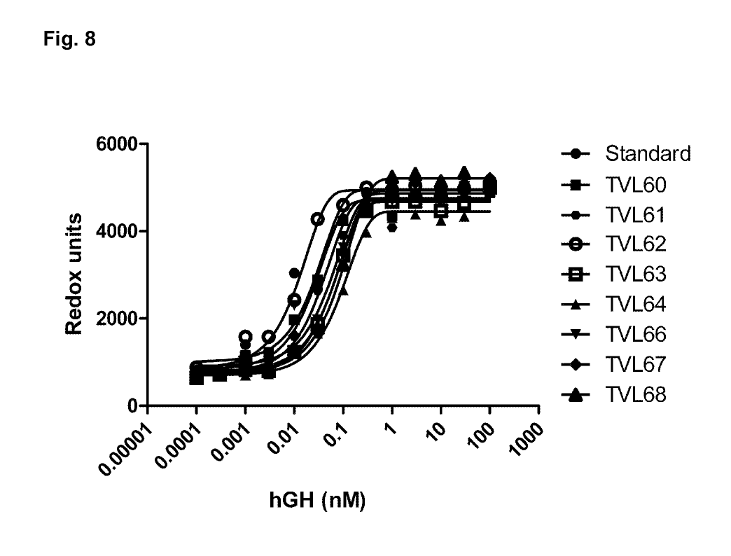

The in vitro activity of 8 human growth hormone mutants with more than one N-glycosylation site was examined with BAF3-GHR cell assay described in Example 6. Results from the activity testing are shown in FIG. 8.

### Example 16

### Purification of Human Growth Hormone with More than One N-glycosylation Site from Mammalian Cell Culture Supernatants

Medium from suspension adapted human embryonal kidney (HEK293F) cells (Freestyle, Invitrogen) transfected with the pTVL64 expression plasmid encoding human growth hormone with the mutations Q49N+E65N+G104N+R127N+E129T, pTVL66 encoding human growth hormone with the mutations Q49N+E65N+S71N+L73T+G104N+R127N+E129T, or pTVL67 encoding human growth hormone with the mutations Q49N+E65N+S71N+L73T+L93N+G104N+R127N+E129T was passed through a 45 μm cellulose acetate filter and a 22 μm polyethersulfone filter (Corning) and afterwards diluted 10-fold in buffer with a final concentration of 25 mM HEPES, pH 7.0 at 4° C. The diluted material was loaded onto a 45 mL (ø=1.8 cm, I=17.5 cm) Source30Q anion exchange column (GE Healthcare) in a process driven by an ÄKTA Explorer equipment (GE Healthcare). Elution of the material from the column was done with 25 mM HEPES and 1 M NaCl, pH 7.0 at 4° C. increasing in concentration from 0 to 20% over 19 column volumes (CV) (840 mL), from 20 to 40% over 10 CV (200 mL) and from 40 to 100% over 5 CV (90 mL). The throughput was registered using UV-absorbance at 254 nm and at 280 nm and was collected in fractions of 10 mL.

### Example 17

### Comparison of the Pharmacokinetic Properties of Human Growth Hormone with More than One N-glycosylation Site with Those of Wild-Type Human Growth Hormone

Recombinant wild-type human growth hormone and human growth hormone with the mutations Q49N+E65N+G104N+R127N+E129T (TVL64), Q49N+E65N+S71N+L73T+G104N+R127N+E129T (TVL66), or Q49N+E65N+S71N+L73T+L93N+G104N+R127N+E129T (TVL67) were diluted in buffer consisting of 20 mg/ml glycine, 2 mg/ml mannitol, 2.4 mg/ml NaHCO3, pH adjusted to 8.2 to a final concentration of 100 nmol/ml. 0.1 ml corresponding to 10 nmol of each compound was administered intravenously via a tail vein (IV) or subcutaneously in the back of the neck to six male Sprague Dawley rats each. The Sprague Dawley rats were weighing approximately 200-250 g.

Blood samples were drawn 5 minutes, 30 minutes and 1, 2, 4, 8, 18, 24, 30, 48, 72, and 96 hours after dosing. 0.3 ml blood samples were drawn as tail vein puncture using a 23 G needle. Blood samples were collected in test tubes containing 8 mM EDTA. Blood samples were kept on ice for a maximum of 20 minutes before centrifugation (1500×g, 4° C., 10 min.). 150 μl plasma was collected from each blood sample, transferred to a test tube and placed on dry ice. Frozen plasma samples were stored at −20° C. until analysis for the content of human growth hormone antigen using compound specific standard curves.

Human growth hormone analogue concentrations were determined by Luminescence Oxygen Channelling Immunoassay (LOCI), which is a homogenous bead based assay. LOCI reagents include two latex bead reagents and biotinyl-mAb 20GS10, which is one part of the sandwich. One of the bead reagents is a generic reagent (donor beads) and is coated with streptavidin and contains a photosensitive dye. The second bead reagent (acceptor beads) is coated with an antibody making up the sandwich. During the assay, the three reactants combine with analyte to form a bead-aggregate-immune complex. Illumination of the complex releases singlet oxygen from the donor beads which channels into the acceptor beads and triggers chemiluminescence which is measured in the EnVision plate reader. The amount of light generated is proportional to the concentration of hGH derivative. 2 μL 40× in LOCI buffer diluted sample/calibrator/control is applied in 384-well LOCI plates. 15 μL of a mixture of biotinyl-mAb 20GS10 and mAb 10G05/M94169 anti-(hGH) conjugated acceptor-beads is added to each well (21-22° C.). The plates are incubated for 1 h at 21-22° C. 30 μL streptavidin coated donor-beads (67 μg/mL) is added to each well and all is incubated for 30 minutes at 21-22° C. The plates are read in an Envision plate reader at 21-22° C. with a filter having a bandwidth of 520-645 nm after excitation by a 680 nm laser. The total measurement time per well is 210 ms including a 70 ms excitation time. The limit of detection for the N-glycosylated human growth hormone analogues were 199, 80 and 350 pM respectively.

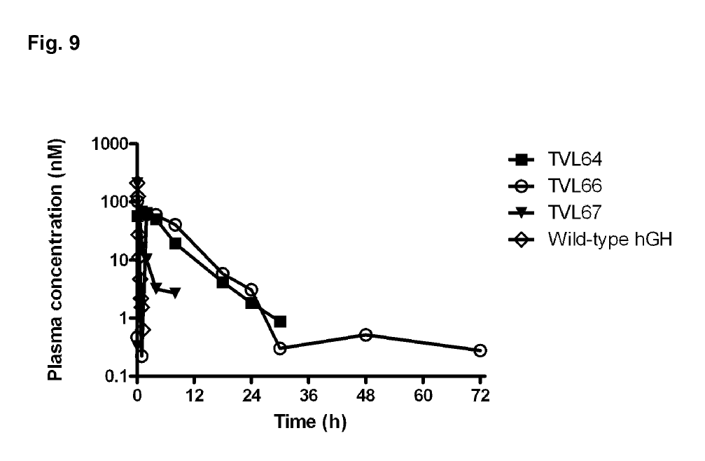

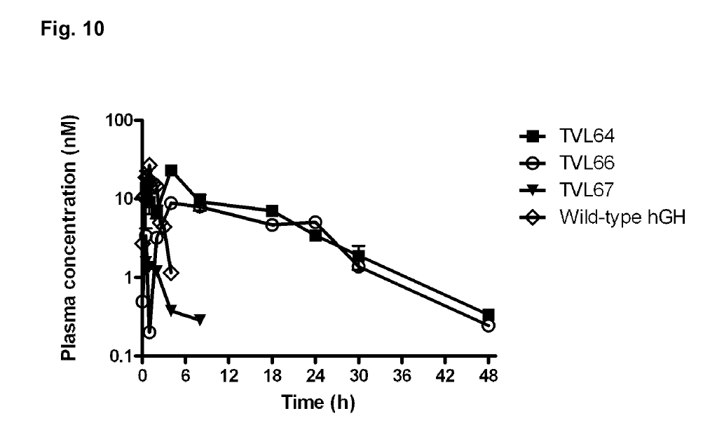

The mean growth hormone antigen concentrations versus time after intravenous dosing are shown in FIG. 9. The mean growth hormone antigen concentrations versus time after subcutaneous dosing are shown in FIG. 10. The estimated pharmacokinetic parameters after intravenous administration are listed in Table 13. The estimated pharmacokinetic parameters after subcutaneous administration are listed in Table 14.

The pharmacokinetic data of human growth hormone with the mutations Q49N+E65N+G104N+R127N+E129T (TVL64) or Q49N+E65N+S71N+L73T+G104N+R127N+E129T (TVL66) showed increased exposure in terms of dose corrected area under the plasma concentration-time curve (AUC), reduced clearance and increased plasma in vivo half-life compared to wild-type human growth hormone in Sprague Dawley rats. The results of mutation Q49N+E65N+S71N+L73T+L93N+G104N+R127N+E129T (TVL67) indicate the same trend as the other mutations; however the sparse data especially after the subcutaneous administration prevented a firm pharmacokinetic conclusion.

TABLE 13
Pharmacokinetic parameters in intravenously dosed Sprague Dawley rats

| | | Terminal | | Mean |
|---|---|---|---|---|
| | AUC/Dose | half-life | Clearance | Residence |
| Compound | (h/L) | (h) | (L/h) | Time (h) |
| Wild-type human growth hormone | 4.23 | 0.23 | 0.237 | 0.15 |
| Q49N + E65N + G104N + R127N + E129T | 50.5 | 4.9 | 0.0198 | 6.3 |
| variant (TVL64) | | | | |
| Q49N + E65N + S71N + L73T + G104N + | 71.3 | 3.3 | 0.0140 | 7.6 |
| R127N + E129T variant (TVL66) | | | | |
| Q49N + E65N + S71N + L73T + L93N + G104N + | 13.0 | 1.3 | 0.0771 | 1.5 |
| R127N + E129T variant | | | | |
| (TVL67) | | | | |

TABLE 14
Pharmacokinetic parameters in subcutaneously dosed Sprague Dawley rats

| | | | | Mean |
|---|---|---|---|---|
| | AUC/Dose | Terminal | Clearance | Residence |
| Compound | (h/L) | half-life (h) | (L/h) | Time (h) |
| Wild-type human growth | 3.33 | 0.58 | 0.300 | 1.5 |
| hormone | | | | |
| Q49N + E65N + G104N + R127N + E129T | 25.4 | 6.9 | 0.0394 | 13.0 |
| variant (TVL64) | | | | |
| Q49N + E65N + S71N + L73T + G104N + | 17.3 | 5.8 | 0.0578 | 15.0 |
| R127N + E129T variant (TVL66) | | | | |
| Q49N + E65N + S71N + L73T + L93N + | 0.65 | 3.2 | 1.55 | 4.8 |
| G104N + R127N + E129T variant | | | | |
| (TVL67) | | | | |

1. A human growth hormone variant, wherein said variant comprises an amino acid sequence comprising one or more N-glycosylation motifs (N-X-S/T), which are not present in the wild type human growth hormone.

2. A human growth hormone variant according to claim 1, wherein the molecular weight of the variant is increased compared to wild-type human growth hormone.

3. A human growth hormone variant according to claim 1, wherein the activity of the variant is substantially the same as the activity wild-type human growth.

4. A human growth hormone variant according to claim 1, wherein the in vivo circulatory half-life of the human growth hormone variant is prolonged compared to wild-type human growth hormone.

5. A human growth hormone variant according to claim 1, wherein at least one of said N-glycosylation motifs (N-X-S/T) not present in the wild type human growth hormone have been generated by introducing one or more mutation(s)/mutation pair(s) selected from the group consisting of: K41N, Q49N, S55N, E65T, E65N, E65S, Q69N, E74S, E74T, R77N, I83N, L93N, A98N, L101S, L101T, G104N, S106N, Y111S, Y111T, I121N, D130N, P133N, K140N, T142N, G161S, G161T, E186N, R19N+H21S/T, A34N+I36S/T, L45N+N47S/T, I58N+P59F, S62N+R64S/T, S71N+L73S/T, K115N+L117S/T, R127N+E129S/T, L128N+D130S/T and T175N+L177S/T.

6. A human growth hormone variant according to claim 5, wherein at least one of said N-glycosylation motifs (N-X-S/T) not present in the wild type human growth hormone have been generated by introducing one or more mutation(s)/mutation pair(s) selected from the group consisting of: K41N, Q49N, E65T, E65N, Q69N, E74T, R77N, I83N, L93N, A98N, L101T, G104N, S106N, Y111T, I121N, D130N, P133N, K140N, T142N, T148N, G161T, E186N, or R19N+H21S, A34N+I36S, L45N+N47S, I58N+P59F, S62N+R64T, S71N+L73T, K115N+L117T, R127N+E129T, L128N+D130T and T175N+L177S.

7. A human growth hormone variant according to claim 6, wherein at least one of said N-glycosylation motifs (N-X-S/T) not present in the wild type human growth hormone have been generated by introducing one or more mutation(s)/mutation pair(s) selected from the group consisting of: K41N, Q49N, E65T, E65N, E74T, L93N, A98N, L101T, G104N, Y111T, P133N, K140N, G161T, E186N, R19N+H21S, I58N+P59F, S62N+R64T, S71N+L73T, R127N+E129T and L128N+D130T.

8. A human growth hormone variant according to claim 5, wherein at least one of said N-glycosylation motifs (N-X-S/T) not present in the wild type human growth hormone have been generated by introducing a mutation selected from the group consisting of: S55N, Q69N, E74S, E74T, R77N, I83N, L93N, A98N, L101S, L101T, G104N, S106N, Y111S, Y111T, I121N, D130N, K140N, T142N, G161S, G161T and E186N.

9. A human growth hormone variant according to claim 5, wherein at least one of said N-glycosylation motifs (N-X-S/T) not present in the wild type human growth hormone have been generated by introducing one or more mutation(s)/mutation pair(s) selected from the group consisting of: Q49N, E65N, L93N, A98N, L101T, G104N, S71N+L73T and R127N+E129T.

10. A human growth hormone variants according to claim 1 including one or more mutations selected from the following sets of mutation(s)/mutation pair(s):
a. Q49N and R127N+E129T,
b. Q49N, E65N and G104N,
c. Q49N, L93N and R127N+E129T,
d. Q49N, E65N, L93N and G104N,
e. Q49N, E65N, G104N and R127N+E129T,
f. Q49N, E65N, S71N+L73T, G104N and R127N+E129T,
g. Q49N, E65N, S71N+L73T, L93N, G104N and R127N+E129T,
h. Q49N, E65N, S71N+L73T, L93N, A98N, G104N and R127N+E129T,
i. S71N+L73T, L93N, A98N and G104N,
j. L93N, G104N and R127N+E129T and
k. S71N+L73T, L93N, G104N and R127N+E129T
l. L93N, A98N, L101T and G104N,
m. L93N, A98N and G104N and
n. L93N, L101T and G104N.

11. A human growth hormone variants according to claim 1 including one or more chemical modifications or additional mutations.

12. A nucleic acid molecule encoding a human growth hormone variant according to claim 1.

13. A method for preparing an N-glycosylated human growth hormone variant, which method comprises the recombinant expression of a nucleic acid molecule according to claim 12 in a eukaryotic cell.

14. An N-glycosylated human growth hormone variant, which N-glycosylated human growth hormone variant is a human growth hormone variant according to claim 1 which has been glycosylated with one or more N-glycans wherein said N-glycans have been attached to one or more of the N-glycosylation motif(s) (N-X-S/T) in said human growth hormone variant, which N-glycosylation motif(s) are not present in the wild type human growth hormone.

15. A preparation comprising an N-glycosylated human growth hormone variant according to claim 14, wherein at least 50% of the growth hormone variant is N-glycosylated.

16. A preparation comprising an N-glycosylated human growth hormone variant according to claim 14, wherein at least 50% of the N-glycans comprise at least one sialic acid moiety.

17. A method for preparing a pharmaceutical composition comprising a N-glycosylated human growth hormone variant according to claim 14, which method comprises the steps of
a. recombinantly expressing a nucleic acid molecule according to claim 8 in a host cell capable of performing N-glycosylation,
b. purifying the N-glycosylated human growth hormone variant,
c. preparing a pharmaceutically acceptable formulation comprising the purified N-glycosylated human growth hormone variant from step b.

18. A pharmaceutical composition comprising an N-glycosylated human growth hormone variant according to claim 14 and a pharmaceutically acceptable carrier.

19. A method of treating a mammal in need of human growth hormone, said method comprising administering to the mammal a therapeutically effective amount of a pharmaceutical composition according to claim 18.


SEQUENCE LISTING (66 sequences)

SEQ ID NO: 1 - protein, 191 aa, Homo sapiens [sequence omitted]

SEQ ID NO: 2 - DNA, 43 bp, Artificial Sequence
  Synthetic Sequence
gcaacagagaagagacccagaataagagcaacctggaactgcg

SEQ ID NO: 3 - DNA, 39 bp, Artificial Sequence
  Synthetic Sequence
gcaacctggaactgctgaatatctctctgctgctgatcc

SEQ ID NO: 4 - DNA, 36 bp, Artificial Sequence
  Synthetic Sequence
ggatctctctgctgctgaatcagagctggctggaac

SEQ ID NO: 5 - DNA, 41 bp, Artificial Sequence
  Synthetic Sequence
ctggaacccgtgcagttcaatagaagcgtgttcgccaacag

SEQ ID NO: 6 - DNA, 40 bp, Artificial Sequence
  Synthetic Sequence
gttcctgagaagcgtgttcaataacagcctggtgtacggc

SEQ ID NO: 7 - DNA, 30 bp, Artificial Sequence
  Synthetic Sequence
gtgttcgccaacagcacggtgtacggcgcc

SEQ ID NO: 8 - DNA, 34 bp, Artificial Sequence
  Synthetic Sequence
caacagcctggtgtacaacgccagcgacagcaac

SEQ ID NO: 9 - DNA, 28 bp, Artificial Sequence
  Synthetic Sequence
ggtgtacggcgccaacgacagcaacgtg

SEQ ID NO: 10 - DNA, 32 bp, Artificial Sequence
  Synthetic Sequence
gcgacagcaacgtgaccgacctgctgaaggac

SEQ ID NO: 11 - DNA, 29 bp, Artificial Sequence
  Synthetic Sequence
cctggaagaaggcaaccagaccctgatgg

SEQ ID NO: 12 - DNA, 32 bp, Artificial Sequence
  Synthetic Sequence
cggccagatcttcaatcagacctacagcaagt

SEQ ID NO: 13 - DNA, 38 bp, Artificial Sequence
  Synthetic Sequence
gctctgctgaagaactacacgctgctgtactgcttcag

SEQ ID NO: 14 - DNA, 27 bp, Artificial Sequence
  Synthetic Sequence
atgggcaggctggaaaatggcagcccc

SEQ ID NO: 15 - DNA, 33 bp, Artificial Sequence
  Synthetic Sequence
cagtgcagaagcgtgaatgggagctgcggcttc

SEQ ID NO: 16 - DNA, 30 bp, Artificial Sequence; identical to SEQ ID NO: 7
  Synthetic Sequence

SEQ ID NO: 17 - DNA, 33 bp, Artificial Sequence
  Synthetic Sequence
caacagcctggtgtacaacgccagcgacagcaa

SEQ ID NO: 18 - DNA, 40 bp, Artificial Sequence
  Synthetic Sequence
gttcaatagaagcgtgttcaacaacagcacggtgtacaac

SEQ ID NO: 19 - DNA, 40 bp, Artificial Sequence
  Synthetic Sequence
gttcaatagaagcgtgttcaacaacagcctggtgtacaac

SEQ ID NO: 20 - DNA, 37 bp, Artificial Sequence
  Synthetic Sequence
gcctacatccccaaagaacagaattacagctttctgc

SEQ ID NO: 21 - DNA, 37 bp, Artificial Sequence
  Synthetic Sequence
gctttctgcagaaccccaatacctccctgtgcttcag

SEQ ID NO: 22 - DNA, 37 bp, Artificial Sequence
  Synthetic Sequence
ccacccccagcaacagaacggagacccagcagaagag

SEQ ID NO: 23 - DNA, 35 bp, Artificial Sequence
  Synthetic Sequence
cacccccagcaacagaaatgagacccagcagaaga

SEQ ID NO: 24 - DNA, 41 bp, Artificial Sequence
  Synthetic Sequence
ccagcagaagagcaacctgacgctgctgaggatctctctgc

SEQ ID NO: 25 - DNA, 32 bp, Artificial Sequence
  Synthetic Sequence
ctggaagatggcagcaacaggaccggccagat

SEQ ID NO: 26 - DNA, 35 bp, Artificial Sequence
  Synthetic Sequence
ccagatcttcaagcagaactacagcaagttcgaca

SEQ ID NO: 27 - DNA, 35 bp, Artificial Sequence
  Synthetic Sequence
ctacagcaagttcgacaacaacagccacaacgacg

SEQ ID NO: 28 - DNA, 40 bp, Artificial Sequence
  Synthetic Sequence
gccatgctgagggcccacaatctgagccagctggcctttg

SEQ ID NO: 29 - DNA, 52 bp, Artificial Sequence
  Synthetic Sequence
cctttgacacctaccaggaatttgaggaaaactacagccccaaagaacagaa

SEQ ID NO: 30 - DNA, 49 bp, Artificial Sequence
  Synthetic Sequence
atccccaaagaacagaagtacagctttaatcagagcccccagacctccc

SEQ ID NO: 31 - DNA, 38 bp, Artificial Sequence
  Synthetic Sequence
gtgcttcagcgagagcaacttcacccccagcaacagag

SEQ ID NO: 32 - DNA, 38 bp, Artificial Sequence
  Synthetic Sequence
gcatccccacccccaacaacacggaagagacccagcag

SEQ ID NO: 33 - DNA, 42 bp, Artificial Sequence
  Synthetic Sequence
gaagagacccagcagaagaacaacacggaactgctgaggatc

SEQ ID NO: 34 - DNA, 45 bp, Artificial Sequence
  Synthetic Sequence
acgtgtacgacctgctgaatgacaatgaagaaggcatccagaccc

SEQ ID NO: 35 - DNA, 44 bp, Artificial Sequence
  Synthetic Sequence
tccagaccctgatgggcaatctgacggatggcagccccaggacc

SEQ ID NO: 36 - DNA, 44 bp, Artificial Sequence
  Synthetic Sequence
cagaccctgatgggcaggaatgaaactggcagccccaggaccgg

SEQ ID NO: 37 - DNA, 41 bp, Artificial Sequence
  Synthetic Sequence
catggacaaggtggagaacttctcgaggatcgtgcagtgca

SEQ ID NO: 38 - DNA, 20 bp, Artificial Sequence
  Synthetic Sequence
caagtttaaacggatctcta

SEQ ID NO: 39 - DNA, 40 bp, Artificial Sequence
  Synthetic Sequence
gcgaattccctgcaatggccaccggcagcaggaccagcct

SEQ ID NO: 40 - DNA, 40 bp, Artificial Sequence
  Synthetic Sequence
gctgctggccttcggcctgctgtgcctgccctggctgcag

SEQ ID NO: 41 - DNA, 40 bp, Artificial Sequence
  Synthetic Sequence
gaaggatccgcctttccaaccatccccctgagcaggctgt

SEQ ID NO: 42 - DNA, 40 bp, Artificial Sequence
  Synthetic Sequence
tcgacaacgccatgctgagggcccacaggctgcaccagct

SEQ ID NO: 43 - DNA, 40 bp, Artificial Sequence
  Synthetic Sequence
ggcctttgacacctaccaggaatttgaggaagcctacatc

SEQ ID NO: 44 - DNA, 40 bp, Artificial Sequence
  Synthetic Sequence
cccaaagaacagaagtacagctttctgcagaacccccaga

SEQ ID NO: 45 - DNA, 40 bp, Artificial Sequence
  Synthetic Sequence
cctccctgtgcttcagcgagagcatccccacccccagcaa

SEQ ID NO: 46 - DNA, 40 bp, Artificial Sequence
  Synthetic Sequence
cagagaagagacccagcagaagagcaacctggaactgctg

SEQ ID NO: 47 - DNA, 40 bp, Artificial Sequence
  Synthetic Sequence
aggatctctctgctgctgatccagagctggctggaacccg

SEQ ID NO: 48 - DNA, 40 bp, Artificial Sequence
  Synthetic Sequence
tgcagttcctgagaagcgtgttcgccaacagcctggtgta

SEQ ID NO: 49 - DNA, 40 bp, Artificial Sequence
  Synthetic Sequence
cggcgccagcgacagcaacgtgtacgacctgctgaaggac

SEQ ID NO: 50 - DNA, 40 bp, Artificial Sequence
  Synthetic Sequence
ctggaagaaggcatccagaccctgatgggcaggctggaag

SEQ ID NO: 51 - DNA, 40 bp, Artificial Sequence
  Synthetic Sequence
atggcagccccaggaccggccagatcttcaagcagaccta

SEQ ID NO: 52 - DNA, 40 bp, Artificial Sequence
  Synthetic Sequence
cagcaagttcgacaccaacagccacaacgacgacgctctg

SEQ ID NO: 53 - DNA, 40 bp, Artificial Sequence
  Synthetic Sequence
ctgaagaactacgggctgctgtactgcttcagaaaggaca

SEQ ID NO: 54 - DNA, 40 bp, Artificial Sequence
  Synthetic Sequence
tggacaaggtggagaccttcctgaggatcgtgcagtgcag

SEQ ID NO: 55 - DNA, 40 bp, Artificial Sequence
  Synthetic Sequence
aagcgtggaggggagctgcggcttctagtagcaagcttgc

SEQ ID NO: 56 - DNA, 40 bp, Artificial Sequence
  Synthetic Sequence
tagcggccgctcgaggccggcaaggccggatcccccgacc

SEQ ID NO: 57 - DNA, 40 bp, Artificial Sequence
  Synthetic Sequence
cccaaagaacagaagtacagctttctgcagaaccccaata

SEQ ID NO: 58 - DNA, 40 bp, Artificial Sequence
  Synthetic Sequence
cagaaatgagacccagcagaagagcaacctggaactgctg

SEQ ID NO: 59 - DNA, 40 bp, Artificial Sequence
  Synthetic Sequence
cagagaagagacccagcagaagaacaacacggaactgctg

SEQ ID NO: 60 - DNA, 40 bp, Artificial Sequence
  Synthetic Sequence
cagaaatgagacccagcagaagaacaacacggaactgctg

SEQ ID NO: 61 - DNA, 40 bp, Artificial Sequence
  Synthetic Sequence
tgcagttcaatagaagcgtgttcgccaacagcctggtgta

SEQ ID NO: 62 - DNA, 40 bp, Artificial Sequence
  Synthetic Sequence
tgcagttcaatagaagcgtgttcaataacagcctggtgta

SEQ ID NO: 63 - DNA, 40 bp, Artificial Sequence
  Synthetic Sequence
caacgccagcgacagcaacgtgtacgacctgctgaaggac

SEQ ID NO: 64 - DNA, 40 bp, Artificial Sequence
  Synthetic Sequence
ctggaagaaggcatccagaccctgatgggcaatctgacgg

SEQ ID NO: 65 - DNA, 674 bp, Artificial Sequence
  Synthetic sequence
aagcttctgcaatggccaccggcagcaggaccagcctgctgctggccttcggcctgctgtgcctgccctggctgcaggaaggatccgcctttccaaccatccccctgagcaggctgttcgacaacgccatgctgagggcccacaggctgcaccagctggcctttgacacctaccaggaatttgaggaagcctacatccccaaagaacagaagtacagctttctgcagaacccccagacctccctgtgcttcagcgagagcatccccacccccagcaacagagaagagacccagcagaagagcaacctggaactgctgaggatctctctgctgctgatccagagctggctggaacccgtgcagttcctgagaagcgtgttcgccaacagcctggtgtacggcgccagcgacagcaacgtgtacgacctgctgaaggacctggaagaaggcatccagaccctgatgggcaggctggaagatggcagccccaggaccggccagatcttcaagcagacctacagcaagttcgacaccaacagccacaacgacgacgctctgctgaagaactacgggctgctgtactgcttcagaaaggacatggacaaggtggagaccttcctgaggatcgtgcagtgcagaagcgtggaggggagctgcggcttctagctggaattc

SEQ ID NO: 66 - protein, 217 aa, Artificial Sequence
  Synthetic Construct
MATGSRTSLLLAFGLLCLPWLQEGSAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF